SEQUENCE LISTING (59 sequences)

SEQ ID NO: 1 - protein, 288 aa, homo sapiens
FLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNSLWKGPAKLLWKGEGAVVIQDNSDIKVVPRRKAKIIRDYGKQMAGDDCVASRQDED

SEQ ID NO: 2 - DNA, 32 bp, homo sapiens
gtatcaaacaggaattcggtatcccgtacaac

SEQ ID NO: 3 - DNA, 33 bp, homo sapiens
gttgtacgggataccgaattcctgtttgatacc

SEQ ID NO: 4 - protein, 159 aa, homo sapiens
GQVDSSPGIWQLDCTHLEGKVILVAVHVASGYIEAEVIPAETGQETAYFLLKLAGRWPVKTIHTDNGSNFTGATVRAACDWAGIKQEFGIPYNPQSQGVVESMNKELKKIIGQVRDQAEHLKTAVQMAVFIHNKKRKGGIGGYSAGERIVDIIATDIQT

SEQ ID NO: 5 - DNA, 37 bp, homo sapiens
gtacgtgctgcagccgactgggctggtatcaaacagg

SEQ ID NO: 6 - DNA, 38 bp, homo sapiens
gaattcctgtttgataccagcccagtcggctgcagcac

SEQ ID NO: 7 - DNA, 33 bp, homo sapiens
atctggcaactggacgcgactcacctcgagggt

SEQ ID NO: 8 - DNA, 33 bp, homo sapiens
accctcgaggtgagtcgcgtccagttgccagat

SEQ ID NO: 9 - DNA, 28 bp, homo sapiens
ggagatatacatatgcaccaccatcacc

SEQ ID NO: 10 - DNA, 29 bp, homo sapiens
atcatcgatgataagcttcctaggtctgg

SEQ ID NO: 11 - DNA, 17 bp, homo sapiens
aatacgactcactatag

SEQ ID NO: 12 - DNA, 30 bp, homo sapiens
gtgagtcgcgtccaggcaccagatacccgg

SEQ ID NO: 13 - DNA, 32 bp, homo sapiens
ctcgaggtgagtcgcgcacagttgccagatac

SEQ ID NO: 14 - DNA, 33 bp, homo sapiens
ctttaccctcgaggtgacacgcgtccagttgcc

SEQ ID NO: 15 - DNA, 37 bp, homo sapiens
ggataactttaccctcgaggcaagtcgcgtccagttg

SEQ ID NO: 16 - DNA, 29 bp, homo sapiens
aactttaccctcgcagtgagtcgcgtcca

SEQ ID NO: 17 - DNA, 29 bp, homo sapiens
gcaaccaggataacgcaaccctcgaggtg

SEQ ID NO: 18 - DNA, 34 bp, homo sapiens
cagtttcctgaccagtgcaggccgggataacttc

SEQ ID NO: 19 - DNA, 34 bp, homo sapiens
ggatccgttgtcagtgcagatggttttaaccggc

SEQ ID NO: 20 - DNA, 35 bp, homo sapiens
gttggatccgttgcaagtgtggatggttttaaccg

SEQ ID NO: 21 - DNA, 35 bp, homo sapiens
cggtagcaccagtgaagcaggatccgttgtcagtg

SEQ ID NO: 22 - DNA, 33 bp, homo sapiens
caccctgagactgcgggcagtacgggataccga

SEQ ID NO: 23 - DNA, 35 bp, homo sapiens
catagattcaacaacaccgcaagactgcgggttgt

SEQ ID NO: 24 - DNA, 34 bp, homo sapiens
gctctttgttcatagattcgcaaacaccctgaga

SEQ ID NO: 25 - DNA, 34 bp, homo sapiens
gctctttgttcatagagcaaacaacaccctgaga

SEQ ID NO: 26 - DNA, 41 bp, homo sapiens
ccgatgatttttttgagctctttgcacatagattcaacaac

SEQ ID NO: 27 - DNA, 35 bp, homo sapiens
ccgatgatttttttgagctcgcagttcatagattc

SEQ ID NO: 28 - DNA, 34 bp, homo sapiens
cctgaccgatgattttgcagagctctttgttcat

SEQ ID NO: 29 - DNA, 32 bp, homo sapiens
cctgatcacgaacctggcagatgatttttttg

SEQ ID NO: 30 - DNA, 34 bp, homo sapiens
ggttttcaggtgttcagcgcaatcacgaacctga

SEQ ID NO: 31 - DNA, 33 bp, homo sapiens
gccatctgaaccgcgcatttcaggtgttcagcc

SEQ ID NO: 32 - protein, 205 aa, homo sapiens
LEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENK

SEQ ID NO: 33 - DNA, 30 bp, homo sapiens
ttactcagacttatttgtttcatagctcta

SEQ ID NO: 34 - DNA, 30 bp, homo sapiens
gaaattagtgactggatatattaactggat

SEQ ID NO: 35 - DNA, 39 bp, homo sapiens
ccggaattcatgaaagtgttactcagacttatttgtttc

SEQ ID NO: 36 - DNA, 42 bp, homo sapiens
ccgctcgagtcacttctggaaattagtgactggatatattaa

SEQ ID NO: 37 - DNA, 39 bp, homo sapiens
ccgctcgagtcatcatttgttttcctctagagtaataaa

SEQ ID NO: 38 - DNA, 36 bp, homo sapiens
ccgctcgagtcatttgttttcctctagagtaataaa

SEQ ID NO: 39 - DNA, 25 bp, homo sapiens
gaggcagtaagatgaatgtcttacc

SEQ ID NO: 40 - DNA, 26 bp, homo sapiens
ctattgcgtggtaagacattcatctt

SEQ ID NO: 41 - DNA, 33 bp, homo sapiens
atgacaagtatagtgccctctatgcttttcacg

SEQ ID NO: 42 - DNA, 33 bp, homo sapiens
gctgaaaagcatagagggcactatacttgtcat

SEQ ID NO: 43 - DNA, 24 bp, homo sapiens
ctcgagtcatcagttttcctctag

SEQ ID NO: 44 - DNA, 20 bp, homo sapiens
tattccggattattcatacc

SEQ ID NO: 45 - DNA, 21 bp, homo sapiens
cctctacaaatgtggtatggc

SEQ ID NO: 46 - DNA, 25 bp, homo sapiens
taaaattattttacattcacgttcc

SEQ ID NO: 47 - DNA, 27 bp, homo sapiens
cactaaaattatacattcttcacgttc

SEQ ID NO: 48 - DNA, 27 bp, homo sapiens
tgacactaaaatacatttttcttcacg

SEQ ID NO: 49 - DNA, 26 bp, homo sapiens
atttgcagatgaacataaaattattt

SEQ ID NO: 50 - DNA, 28 bp, homo sapiens
aaatatggcttggcaattataacataag

SEQ ID NO: 51 - DNA, 27 bp, homo sapiens
cttaaatatggcgcatgcattataaca

SEQ ID NO: 52 - DNA, 28 bp, homo sapiens
gtttctgcttaaagcaggcttgtgcatt

SEQ ID NO: 53 - DNA, 24 bp, homo sapiens
gggtagtttctgacaaaatatggc

SEQ ID NO: 54 - DNA, 27 bp, homo sapiens
aacgggtagtttacacttaaatatggc

SEQ ID NO: 55 - DNA, 29 bp, homo sapiens
ctgcaacgggtaggcactgcttaaatatg

SEQ ID NO: 56 - DNA, 28 bp, homo sapiens
ctccgtctcctgcacagggtagtttctg

SEQ ID NO: 57 - DNA, 24 bp, homo sapiens
catataagggcaacaaagtcctcc

SEQ ID NO: 58 - DNA, 27 bp, homo sapiens
aaaaaactccatacaagggcacacaag

SEQ ID NO: 59 - DNA, 24 bp, homo sapiens
tttaaaaaaacacatataagggca

(19) United States
(12) Patent Application Publication
(10) Pub. No.: US 2003/0235862 A1
(43) Pub. Date: Dec. 25, 2003

(54) Methods of characterizing molecular interaction sites on proteins

(76) Inventors: Michelle Arkin, San Francisco, CA (US); Brian Cunningham, San Mateo, CA (US); Warren DeLano, San Carlos, CA (US); Raymond Fucini, San Bruno, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/407,416

(22) Filed: Apr. 4, 2003

Related U.S. Application Data
(60) Provisional application No. 60/370,699, filed on Apr. 5, 2002.
(60) Provisional application No. 60/378,168, filed on May 14, 2002.

(51) Int. Cl. G01N 33/53; G01N 33/543

(52) U.S. Cl. 435/7.1; 436/518

(57) ABSTRACT

The present invention is directed to methods of characterizing molecular interaction sites on a biological target molecule. The methods involve contacting a polypeptide with a library of ligand candidates each of which is capable of tethering to the polypeptide at a site of interest, and analyzing the physicochemical properties of the ligand candidates tethering to the polypeptide. The methods herein are useful for discovery of and development of small molecule drugs.

### CROSS-REFERENCE TO RELATED APPLICATIONS

This application claims priority to provisional application Serial No. 60/370,699 filed Apr. 5, 2002 and to provisional application Serial No. 60/378,168 filed May 14, 2002, the entire disclosures of both provisional applications are incorporated herein by reference.

### BACKGROUND OF THE INVENTION

Small molecule drug discovery concerns to a great extent the molecular recognition of biological target molecules. One challenge in structure-based design is that availability and usefulness of structural information of targets is often limited. In structures of protein targets, for example, side chains in the regions of interest are often disordered and/or adaptable; this is especially true of protein-protein interaction sites that have been largely intractable for small molecule drug development to date.

### SUMMARY OF THE INVENTION

The invention is directed to methods for characterizing molecular interaction sites on proteins. The information obtained using these methods aids, for example, in the identification and/or development of pharmaceutical compounds.

### DESCRIPTION OF THE FIGURES

FIG. 1A shows significant enrichments of compounds with certain characteristics that tether to cysteine mutants of HIV integrase, as compared with compounds with those characteristics in the total library, where p is less than or equal to 0.01. For example, significant enrichment by a factor of approximately 5 is observed for compounds comprising halogens (HAL) tethering to Q62C. FIG. 1B shows significant depletions of compounds with certain characteristics that tether to mutants of HIV integrase, as compared with compounds with those characteristics in the total library, where p is less than or equal to 0.01. Significant depletion by greater than a factor of 7 is observed, for example, for compounds with extended groups (EXT) tethering to Q62C.

FIG. 2 shows the number of compounds having a particular chemical feature that conjugate to various cysteine mutants of HIV integrase, individuated by the distance measured in number of intervening bonds between the chemical feature and the tether position. The chemical features are shown on the x-axis, the cysteine mutants are shown on the first y-axis, and the number of compounds with the chemical feature conjugating each respective cysteine mutant is shown on the second y-axis. Distance from the tether position for each chemical feature increases from left to right.

FIG. 3 shows for several HIV integrase mutants the fraction conjugated with individual compounds. The cysteine mutants are on the x-axis and the fraction of each mutant that conjugates the compound is on the y-axis.

FIG. 4A shows significant enrichments of compounds having a physicochemical property tethering to cysteine mutants of IL-1R as compared with the compounds with the physicochemical property in the library. Significant enrichment by a factor of approximately 2 is observed, for example, for compounds with a 66 fused ring system tethering to E11C. FIG. 4B shows significant depletions of compounds having a physicochemical property that tether to cysteine mutants of IL-1R as compared with compounds with those characteristics in the library. Significant depletion (p less than or equal to 0.01) of greater than a factor of 7 is observed, for example, for compounds with basic groups tethering to E11C.

FIG. 5 shows the number of compounds with particular scaffolds or substituents that conjugate to each of two cysteine mutants of IL-1R. In order to be counted in the analysis, the compound had to conjugate at least 30% of the protein when tested individually.

### DETAILED DESCRIPTION OF THE PREFERRED EMBODIMENTS

Definitions

The term “ligand candidate” refers to a compound that possesses or has been modified to possess a reactive group that is capable of forming a covalent bond with a complimentary or compatible reactive group on a target. The reactive group on either the ligand candidate or the target can be masked with, for example, a protecting group.

The phrase “protected thiol” as used herein refers to a thiol that has been reacted with a group or molecule to form a covalent bond that renders it less reactive and which may be deprotected to regenerate a free thiol.

The phrase “reversible covalent bond” as used herein refers to a covalent bond that can be broken, preferably under conditions that do not denature the target. Examples include, without limitation, disulfides, Schiff-bases, thioesters, coordination complexes, boronate esters, and the like.

The phrase “chemically reactive group” is a chemical group or moiety providing a site at which a covalent bond can be made when presented with a compatible or complementary reactive group. Illustrative examples are —SH that can react with another —SH or —SS— to form respectively a disulfide bond or disulfide exchange; an —NH₂ that can react with an activated —COOH to form an amide; an —NH₂ that can react with an aldehyde or ketone to form a Schiff base and the like.

The phrase “site of interest” refers to any site on a target on which a ligand can bind. For example, the site of interest may be an active site, an allosteric site, or a protein-protein interaction site.

The terms “target,” refers to a protein or a plurality of chemical or biological molecules comprising a protein that are capable of forming a complex with one another. The target can be a protein, a portion of a protein, an aggregate of proteins, or an aggregate of protein(s) with other molecules. In a preferred embodiment, the target molecule is a protein or a portion thereof or that comprises two or more amino acids, and which possesses or is capable of being modified to possess a reactive group that is capable of forming a covalent bond with a compound having a complementary reactive group. In the most preferred embodiment, the target has a naturally occurring cysteine, or has been mutated to possess a cysteine. The term “target” is used for both the naturally occurring protein and for the protein that has been mutated to possess a cysteine in one or more positions. The binding of a ligand to the target may be reversible or irreversible. Specific examples of target molecules include polypeptides or proteins, receptors, transcription factors, ligands for receptors, growth factors, cytokines, immunoglobulins, nuclear proteins, signal transduction components, allosteric enzyme regulators, and the like. The target can be obtained in a variety of ways, including isolation and purification from natural source, chemical synthesis, recombinant production and any combination of these and similar methods.

The term “HIV-1 integrase” is used herein in the broadest sense and includes any polypeptide comprising at least the central catalytic core domain of the native-sequence HIV-1 integrase, or a variant. The central catalytic core domain comprises from about amino acid residue 52 to about amino acid residue 210 of SEQ. ID. NO: 1, shown below. The native sequence of HIV-1 integrase is shown below as SEQ ID NO: 1:

FLDGIDKAQDEHEKYHSNWRAMASDFNLPPVVAKEIVASCDKCQLKGEAM
HGQVDCSPGIWQLDCTHLEGKVILVAVHVASGYIEAEVIPAETGQETAYF
LLKLAGRWPVKTIHTDNGSNFTGATVRAACWWAGIKQEFGIPYNPQSQGV
VESMNKELKKIIGQVRDQAEHLKTAVQMAVFIHNFKRKGGIGGYSAGERI
VDIIATDIQTKELQKQITKIQNFRVYYRDSRNSLWKGPAKLLWKGEGAVV
IQDNSDIKVVPRRKAKIIRDYGKQMAGDDCVASRQDED

The “oral availability” of a drug molecule as used herein refers to the fraction of the oral dose reaching systemic circulation.

Preferred Embodiments

Methods described herein utilize tethering to characterize molecular interaction sites of a polypeptide target. Tethering provides information on molecular fragments that bind the target, e.g., their approximate location of binding on the target and their physicochemical properties; the information is applied in the characterization methods described herein.

The tethering method relies upon the formation of a covalent bond between the target and a compound thereby forming a target-compound conjugate. The tethering method is described in U.S. Pat. No. 6,335,155, PCT Publications No. WO 00/00823 and WO 02/42773; Erlanson et al, Proc. Nat. Acad. Sci. USA 97:9367-9372 (2000); and U.S. patent application Ser. No. 10/121,216 filed on Apr. 10, 2002, which are all incorporated herein by reference, and is described briefly below.

In preferred embodiments, the target is a protein and the chemically reactive group is a thiol on a cysteine residue therein. If a site of interest does not include a naturally occurring cysteine residue, then the target can be modified to include a cysteine residue at or near the site of interest. A cysteine is said to be near the site of interest if it is located within 10 Ångstroms from the site of interest, preferably within 5 Ångstroms from the site of interest. Preferred residues for modification are those that are solvent-accessible. Solvent accessibility may be calculated from structural models using standard numeric [Lee, B. & Richards, F. M. J. Mol. Biol 55:379-400 (1971); Shrake, A. & Rupley, J. A. J. Mol. Biol. 79:351-371 (1973)] or analytical [Connolly, M. L. Science 221:709-713 (1983); Richmond, T. J. J. Mol. Biol. 178:63-89 (1984)] methods. For example, a potential cysteine variant is considered solvent-accessible if the combined surface area of the carbon-beta (CB), or sulfur-gamma (SG) is greater than 20 Å² when calculated by the method of Lee and Richards [Lee, B. & Richards, F. M. J. Mol. Biol 55:379-400 (1971)]. This value represents approximately 33% of the theoretical surface area accessible to a cysteine side-chain as described by Creamer et al. [Creamer, T. P. et al. Biochemistry 34:16245-16250 (1995)].

It is also preferred that the residue to be mutated to cysteine, or another thiol-containing amino acid residue, not participate in hydrogen-bonding with backbone atoms or, that at most, it interacts with the backbone through only one hydrogen bond. Wild-type residues where the side-chain participates in multiple (>1) hydrogen bonds with other side-chains are also less preferred. Variants for which all standard rotamers (chi1 angle of −60°, 60°, or 180°) can introduce unfavorable steric contacts with the N, C_{α}, C, O, or C_{β} atoms of any other residue are also less preferred. Unfavorable contacts are defined as interatomic distances that are less than 80% of the sum of the van der Waals radii of the participating atoms. In certain embodiments where the site of interest is a concave region, residues found at the edge of such a site (such as a ridge or an adjacent convex region) are more preferred for mutating into cysteine residues. Convexity and concavity can be calculated based on surface vectors [Duncan, B. S. & Olson, A. J. Biopolymers 33:219-229 (1993)] or by determining the accessibility of water probes placed along the molecular surface [Nicholls, A. et al. Proteins 11:281-296 (1991); Brady, G. P., Jr. & Stouten, P. F. J. Comput. Aided Mol. Des. 14:383-401 (2000)]. Residues possessing a backbone conformation that is nominally forbidden for L-amino acids [Ramachandran, G. N. et al. J. Mol. Biol. 7:95-99 (1963); Ramachandran, G. N. & Sasisekharahn, V. Adv. Prot. Chem. 23:283-437 (1968)] are less preferred targets for modification to a cysteine. Forbidden conformations commonly feature a positive value of the phi angle.

Other preferred variants are those which, when mutated to cysteine and tethered as to comprise -Cys-SSR¹, would possess a conformation that directs the atoms of R¹ towards the site of interest. Two general procedures can be used to identify these preferred variants. In the first procedure, a search is made of unique structures [Hobohm, U. et al. Protein Science 1:409-417 (1992)] in the Protein Databank [Berman, H. M. et al. Nucleic Acids Research 28:235-242 (2000)] to identify structural fragments containing a disulfide-bonded cysteine at position j in which the backbone atoms of residues j−1, j, and j+1 of the fragment can be superimposed on the backbone atoms of residues i−1, i, and i+1 of the target molecule with an RMSD of less than 0.75 squared Ångstroms. If fragments are identified that place the C_{β} atom of the residue disulfide-bonded to the cysteine at position j closer to any atom of the site of interest than the C_{β} atom of residue i (when mutated to cysteine), position i is considered preferred. In an alternative procedure, the residue at position i is computationally “mutated” to a cysteine and capped with an S-Methyl group via a disulfide bond.

In addition to adding one or more cysteines to a site of interest, it may be desirable to delete one or more naturally occurring cysteines (and replacing them with alanines for example) that are located outside of the site of interest. Various recombinant, chemical, synthesis and/or other techniques can be employed to modify a target such that it possesses a desired number of free thiol groups that are available for tethering. Such techniques include, for example, site-directed mutagenesis of the nucleic acid sequence encoding the target polypeptide such that it encodes a polypeptide with a different number of cysteine residues. Particularly preferred is site-directed mutagenesis using polymerase chain reaction (PCR) amplification [see, for example, U.S. Pat. No. 4,683,195 issued Jul. 28, 1987; and Current Protocols In Molecular Biology, Chapter 15 (Ausubel et al., ed., 1991)]. Other site-directed mutagenesis techniques are also well known in the art and are described, for example, in the following publications: Ausubel et al., supra, Chapter 8; Molecular Cloning: A Laboratory Manual., 2nd edition (Sambrook et al., 1989); Zoller et al., Methods Enzymol. 100:468-500 (1983); Zoller & Smith, DNA 3:479-488 (1984); Zoller et al., Nuceic. Acids Res., 10:6487 (1987); Brake et al., Proc. Natl. Acad. Sci. USA 81:4642-4646 (1984); Botstein et al., Science 229:1193 (1985); Kunkel et al., Methods Enzymol. 154:367-82 (1987), Adelman et al., DNA 2:183 (1983); and Carter et al., Nucleic. Acids Res., 13:4331 (1986). Cassette mutagenesis [Wells et al., Gene, 34:315 (1985)], and restriction selection mutagenesis [Wells et al., Philos. Trans. R. Soc. London SerA, 317:415 (1986)] may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

Once the target-compound conjugate is form ed, it can be detected using a number of methods. In one embodiment, mass spectroscopy is used. The target-compound conjugate can be detected directly in the mass spectroscopy or the target compound conjugate can be fragmented prior to detection. Alternatively, the compound can be liberated within the mass spectrophotometer and subsequently identified.

MS detects molecules based on mass-to-charge ratio (m/z) and thus can resolve molecules based on their sizes [reviewed in Yates, Trends Genet. 16: 5-8 (2000)]. A mass spectrometer first converts molecules into gas-phase ions, then individual ions are separated on the basis of m/z ratios and are finally detected. A mass analyzer, which is an integral part of a mass spectrometer, uses a physical property [e.g. electric or magnetic fields, or time-of-flight (TOF)] to separate ions of a particular m/z value that then strikes the ion detector. Mass spectrometers are capable of generating data quickly and thus have a great potential for high-throughput analysis. Mass spectroscopy may be employed either alone or in combination with other means for detection or identifying the compounds covalently bound to the target. Further descriptions of mass spectroscopy techniques include Fitzgerald and Siuzdak, Chemistry & Biology 3: 707-715 (1996); Chu et al., J. Am. Chem. Soc. 118: 7827-7835 (1996); Siudzak, Proc. Natl. Acad. Sci. USA 91: 11290-11297 (1994); Burlingame et al., Anal. Chem. 68: 599R-651R (1996); Wu et al., Chemistry & Biology 4: 653-657 (1997); and Loo et al., Am. Reports Med. Chem. 31: 319-325 (1996).

The target-compound conjugate can be identified using other means. For example, one can employ various chromatographic techniques such as liquid chromatography, thin layer chromatography and the like for separation of the components of the reaction mixture so as to enhance the ability to identify the covalently bound molecule. Such chromatographic techniques can be employed in combination with mass spectroscopy or separate from mass spectroscopy. One can also couple a (fluorescently, radioactively, or otherwise) labeled probe to the liberated compound so as to facilitate its identification using any of the above techniques. In yet another embodiment, the formation of the new bonds liberates a labeled probe, which can then be monitored. A simple functional assay, such as an ELISA or enzymatic assay can also be used to detect binding when binding occurs in an area essential for what the assay measures. Other techniques that may find use for identifying the organic compound bound to the target molecule include, for example, nuclear magnetic resonance (NMR), surface plasmon resonance (e.g., BIACORE), capillary electrophoresis, X-ray crystallography, and the like, all of which will be well known to those skilled in the art.

The tethering experiments are preferably done under reducing conditions, most preferably where the ratio of the thiol-containing protein in the reduced state vs. in the oxidized state is about 90/10. In the examples described herein, the range of reducing agent used was between about 0.5 and about 4.0 mM. Illustrative examples of suitable reducing agents include cysteine, cysteamine, dithiothreitol, dithioerythritol, glutathione, 2-mercaptoethanol, 3-mercaptopropionic acid, a phosphine such as tris-(2-carboxyethyl-phosphine) (“TCEP”), or sodium borohydride.

In one aspect, the invention is directed to a method for characterizing a molecular interaction site on a protein, the method comprising:

a) contacting a polypeptide comprising a first chemically reactive group at a site of interest with a library of ligand candidates wherein each ligand candidate comprises a second chemically reactive group that is capable of forming a reversible covalent bond with the first chemically reactive group;

b) forming a reversible covalent bond between the first chemically reactive group and the second chemically reactive group of a plurality of ligand candidates;

c) detecting the ligand candidates that formed the reversible covalent bond;

d) identifying a physicochemical property of at least one of the ligand candidates that formed the reversible covalent bond;

e) determining the number of ligand candidates that formed the reversible covalent bond having the physicochemical property; and

f) determining the number of ligand candidates in the library having the physicochemical property.

In the methods described herein, a library of ligand candidates must contain at least two ligand candidates. In one embodiment, the library contains at least 10 ligand candidates. In another embodiment, the library contains at least 50 ligand candidates. In another embodiment, the library contains at least 500 ligand candidates. In another embodiment, the library contains at least 1000 ligand candidates. In another embodiment, the library contains at least 10,000 ligand candidates. In cases of large libraries, the ligand candidates can be screened in smaller pools. When MS is to be used for identification of the hits, it is preferable to create pools where each ligand candidate has a unique mass.

A site of interest is any site on the polypeptide to which a ligand can bind. The nature of the site or sites of interest for a given polypeptide depends in part upon the function of the polypeptide. For example, if the polypeptide is an enzyme, a site of interest is an active site. Other sites of interest on an enzyme include specific binding sites for molecules other than the substrate, such as cofactors or prosthetic groups. For example, in HIV integrase both the substrate-binding site and the cacodylate-binding site are sites of interest. Similarly, a site of interest on a receptor includes, for example, the ligand binding site and any regulatory binding sites.

Notably, the methods herein are independent of known ligands and three-dimensional structures of the polypeptide to determine a site of interest. For example, in cases of orphan receptors where the polypeptide has no known binding sites, sites of interest are established by systematic investigation of whether a particular site forms a reversible covalent bond with at least one of the ligand candidates. Depending on the information available, a region or the entire surface may be systematically explored. Alternatively, biochemical information derived from the effect of site-directed mutagenesis on protein function can be used in determining which residues of the target are at a site of interest. For example, alanine-scanning mutagenesis provides information on the regions of a polypeptide important for contact with another polypeptide. Other methods include use of information from site-directed mutagenesis such as its effect on enzyme activity. Other types of structural and nonstructural information can also be used. For example, NMR spectra in the presence and absence of the small molecule can indicate which resonances are perturbed upon binding of the polypeptide by a small molecule. Residues corresponding to these resonances would also be considered to be at a site of interest. However, when a three-dimensional structure is available, the structure guides the selection of a site of interest.

In a preferred embodiment of the method, the residues at the site of interest either comprise or are modified to comprise a thiol group as the first chemically reactive group. This modification can be accomplished by site-directed mutagenesis of a noncysteine residue to a cysteine.

In another variation of the methods described herein, the statistical difference between the incidence of ligand candidates with a physicochemical property in a library and the incidence of ligand candidates with the property in a subset of members forming a reversible covalent bond with a polypeptide is determined. In one embodiment, the method comprises:

a) contacting a polypeptide comprising a first chemically reactive group at a site of interest with a library of ligand candidates wherein each ligand candidate comprises a second chemically reactive group that is capable of forming a reversible covalent bond with the first chemically reactive group;

b) forming a reversible covalent bond between the first chemically reactive group and the second chemically reactive group of a plurality of the ligand candidates;

c) detecting the ligand candidates that formed the reversible covalent bond;

d) identifying a physicochemical property of at least one of the ligand candidates that formed the reversible covalent bond;

e) determining the number of ligand candidates that formed the reversible covalent bond having the physicochemical property;

f) determining the number of ligand candidates in the library having the physicochemical property; and

g) comparing the prevalence of ligand candidates with the physicochemical property that form the reversible covalent bond with the prevalence of ligand candidates with the physicochemical property in the library.

In another embodiment, the method further comprises measuring the statistical significance of the comparison. In another embodiment, the method further comprises characterizing the molecular interaction site by significant enrichment of the prevalence of the physicochemical property among ligand candidates forming the reversible covalent bond. In still another embodiment, the method further comprises characterizing the molecular interaction site by significant depletion of the prevalence of the physicochemical property among ligand candidates forming the reversible covalent bond. In another embodiment, the method further comprises characterizing the molecular interaction site by a pattern of significant enrichment and significant depletion of the prevalence of the physicochemical property among ligand candidates forming the reversible covalent bond.

In another aspect, the invention is directed to a method comprising:

a) contacting a polypeptide comprising a first chemically reactive group at a site of interest with a library of ligand candidates wherein each ligand candidate comprises a second chemically reactive group that is capable of forming a reversible covalent bond with the first chemically reactive group;

b) forming a reversible covalent bond between the first chemically reactive group and the second chemically reactive group of a plurality of the ligand candidates;

c) detecting the ligand candidates that formed the reversible covalent bond;

d) identifying a physicochemical property of at least one of the ligand candidates that formed the reversible covalent bond;

e) calculating the statistical significance of the incidence of ligand candidates having the physicochemical property in the plurality; and

f) repeating each of step a) through step e) for at least one positional variant of the polypeptide, the positional variant comprising an identical first chemically reactive group at a different sequence location.

In one embodiment of the characterization method, a set of positional variants is contacted with the library of ligand candidates, wherein each positional variant in the set possesses the first chemically reactive group at a different location. The set of positional variants contains at a minimum two variants. For polypeptide where a set of residues comprising the site of interest is well defined, the set of positional variants may be designed so as to focus on the site of interest. For polypeptides where the set of residues comprising the site of interest is not well defined or not yet known, the set of positional variants may be chosen so that they are dispersed throughout the sequence with the aim of systematically representing the sequence.

Just as the characterization methods herein do not rely upon three-dimensional structural information to determine if a polypeptide residue is at a site of interest, they do not depend upon three-dimensional structural information for interpretation of results. For example, even in the absence of any structural information, it is known a priori for any polypeptide that residues that are directly adjacent in sequence will also be proximal to one another in their three-dimensional location. For some polypeptides, information may also be available about metal coordination. If it is known that several residues coordinate a single metal ion, then it can be inferred that they are in spatial proximity to one another, even if they are not directly adjacent in the primary sequence. Importantly, by use of the method described herein, similar patterns of statistically significant enrichment and/or statistically significant depletion of particular ligand candidate physicochemical properties may become apparent for residues that are not adjacent in primary sequence. The similar patterns may be used to define or further refine a site of interest. Alternatively, three-dimensional structural considerations can be used to interpret results obtained from the characterization methods described herein, such as, for example, by mapping areas of the polypeptide having similar patterns of enrichments and/or depletions of particular ligand candidate physicochemical properties among ligand candidates binding to the area.

In one embodiment of the methods described herein the physicochemical property is the presence of a chemical feature. Of particular interest are chemical features present in drug molecules. A method of characterizing molecular interaction sites on proteins is particularly useful in light of the fact that a limited set of structures, substructures, and functional groups predominate among known drugs. Such characterizations are useful for the discovery and development of small molecules that interact non-covalently with these sites. The spatial forms of drugs may be defined, for example, in terms of rings, linkers connecting the rings, and peripheral functionalities.

In another embodiment of the method, the chemical feature is a molecular shape. A molecular shape is a two-dimensional map of the rings and linkers, not taking into account atom type, hybridization, or bond order [Bemis & Murcko J. Med. Chem. 39, 2887-2893 (1996)]. In another embodiment, the physicochemical property is selected from the group consisting of the following molecular shapes:

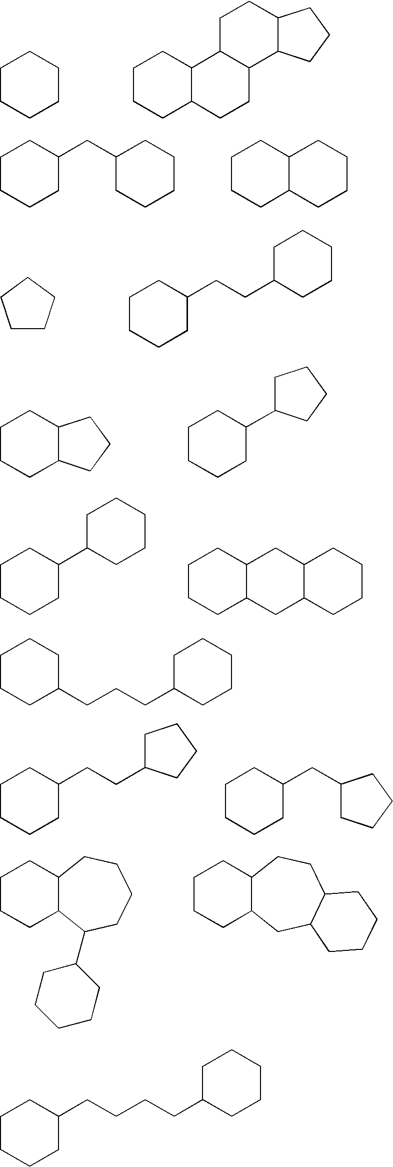

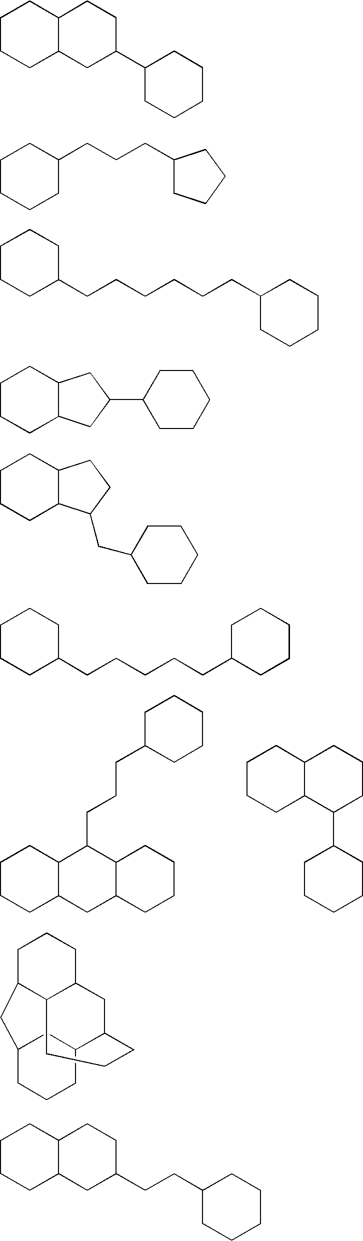

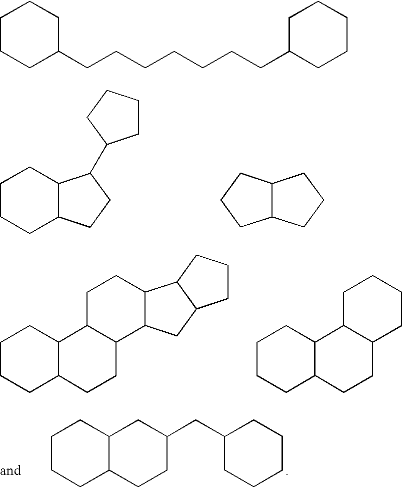

These shapes are the 32 most frequently occurring molecular shapes among known drugs, and can be used to describe approximately half of drugs in the Comprehensive Medicinal Chemistry database (CMC) [Bemis & Murcko J. Med. Chem. 39, 2887-2893 (1996)].

In another embodiment, the physicochemical property is an atomic skeleton. An atomic skeleton is a more detailed molecular shape that takes into account not only the two-dimensional map of the rings and the linkers, but additionally atom types, hybridization and bond order of the component atoms of the molecular shape [Bemis & Murcko J. Med. Chem. 39, 2887-2893 (1996)]. In another embodiment, the chemical feature is selected from group consisting of the following atomic skeletons:

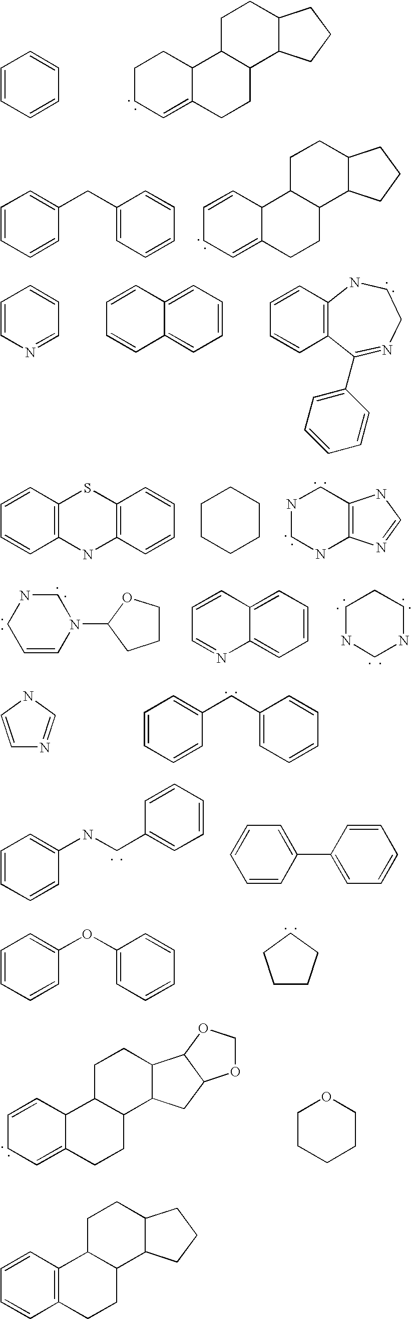

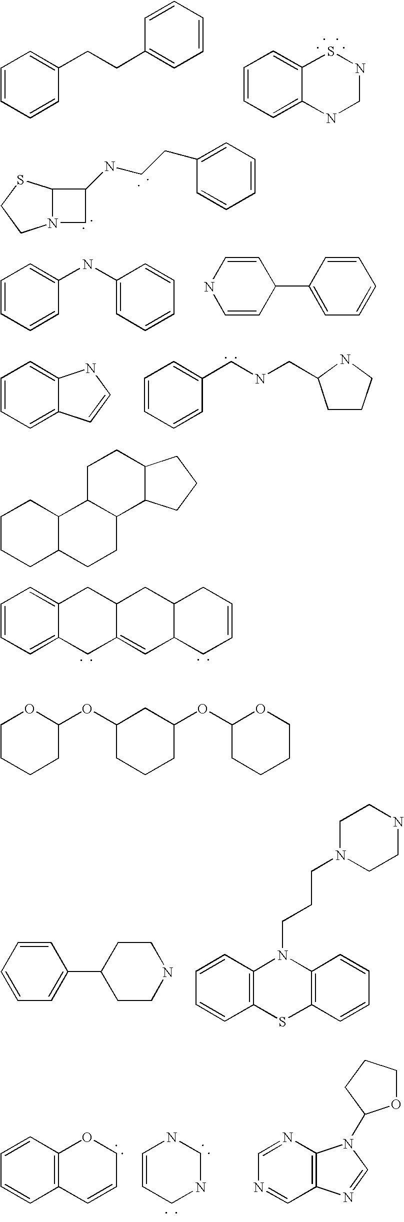

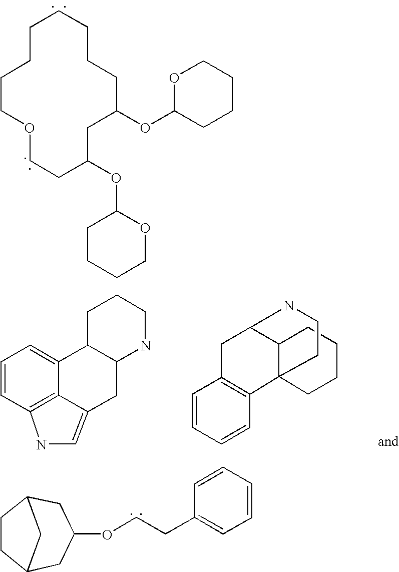

These atomic skeletons are the 41 most commonly occurring atomic skeletons in known drugs, and can account for approximately one-fourth of drugs in the CMC [Bemis & Murcko J. Med. Chem. 39, 2887-2893 (1996)].

In yet another embodiment the chemical feature is selected from the group consisting of the following atomic skeletons:

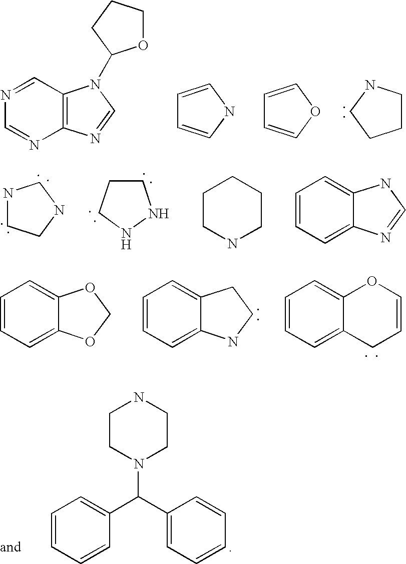

These atomic skeletons are also commonly occurring in drug molecules [Fejzo, el al., Chem. Biol. 6, 755-769 (1999)].

Whereas molecular shapes and atomic skeleton representations depict the framework of a drug molecule as a whole, a drug molecule may also be analyzed in terms of components of its framework. That is, the chemical feature can also be a substructure found to be common to known drugs. For example, the benzene ring is the most prevalent drug substructure, more abundant than all heterocyclic rings combined [Bemis & Murcko J. Med. Chem. 39, 2887-2893 (1996); Ghose, et al. J. Comb. Chem. 1, 55-68 (1999)]. Additionally, among drugs in the CMC, aliphatic heterocycles outnumber aromatic heterocycles by about 2-fold. Among nitrogen-containing heterocycles in the CMC, pyridine is the most prevalent. In a specific embodiment of the method described herein, the chemical feature is the presence of a benzene ring. In another embodiment, the chemical feature is the presence of a heterocyclic ring. In another embodiment, the chemical feature is the presence of a non-aromatic heterocyclic ring. In another embodiment, the chemical feature is the presence of an aromatic heterocyclic ring. In another embodiment, the chemical feature is the presence of a nitrogen-containing heterocyclic ring. In another embodiment, the chemical feature is the presence of a pyridine ring.

In another embodiment, the chemical feature is the presence of a peripheral functionality. Peripheral functionalities are groups attached to a ring or a linker at only one point, and take atom type, hybridization, and bond order of component atoms into account. In another embodiment, the chemical feature is selected from the group consisting of the following peripheral functionalities, commonly found in drugs:

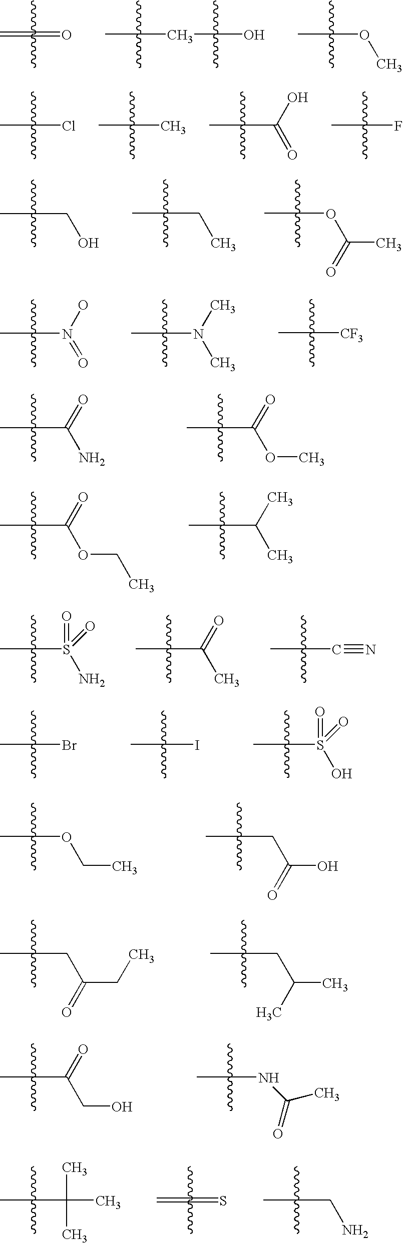

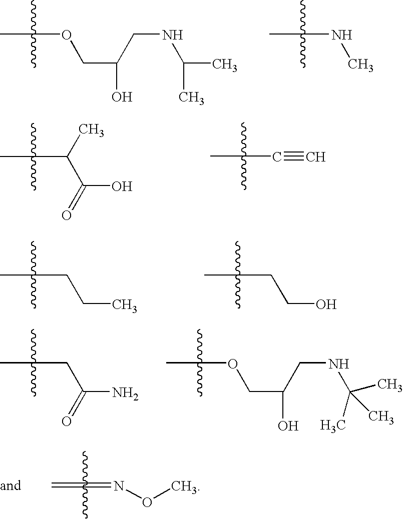

The wavy line divides the bond attaching the peripheral functionality to the ring or the linker.

In another embodiment, the chemical feature is the presence of an attached moiety. An attached moiety is similar to a peripheral functionality in that it takes into account atom type, hybridization, and bond order; an attached moiety additionally takes into account the type of atom on the ring or linker to which the moiety is connected [Bemis & Murcko J. Med. Chem. 42 5095-5099 (1999)], as well as the order of the bond connecting the atom and the attached moiety. In another embodiment, the chemical feature is selected from the group consisting of the following attached moieties taking the atom of attachment into account:

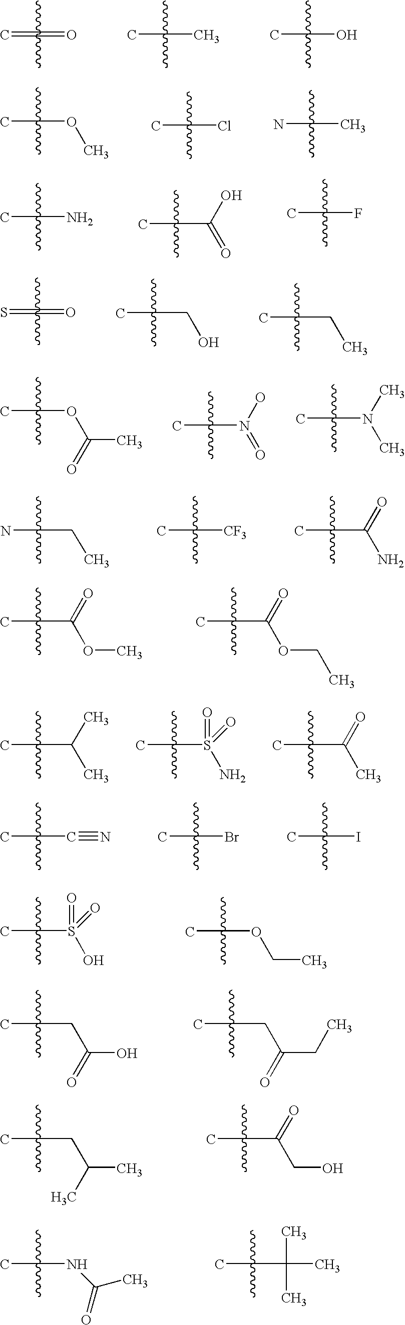

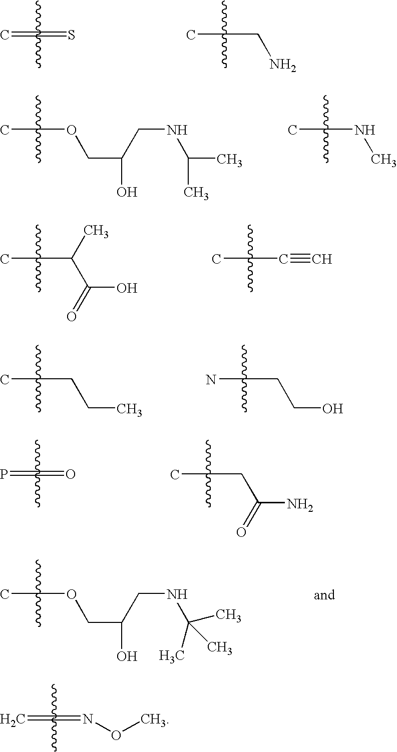

A wavy line divides the bond of attachment, and the atom of attachment on the ring or linker is shown at left-hand side of the bond of attachment. Exclusive of carbonyls, shown in the first row above, there are 15,000 occurrences of attached moieties in drugs in the CMC. Of these, nearly three-quarters are from the group of the 20 most frequently occurring attached moieties [Bemis & Murcko J. Med. Chem. 42 5095-5099 (1999)].

In another embodiment of the methods herein, the chemical feature is chosen from the 20 most frequently occurring attached moieties following:

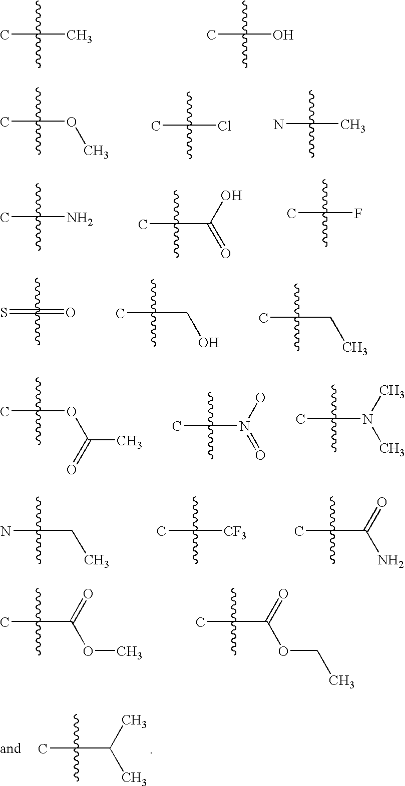

In another embodiment, the chemical feature is the presence of an amine, carboxamide or alcohol. In another embodiment, the chemical feature is the presence of a keto group or a carboxy ester. In another embodiment, the physicochemical property is the presence of a carboxyl acid group or amines. Each of the aforementioned substituents is among the most prevalent in drug molecules [Ghose, et al. J. Comb. Chem. 1, 55-68 (1999)].

Interestingly, some classes of drugs show differences in the substituents they contain. For example, carboxyl acid groups are nearly absent in the classes of drugs acting on the central nervous system (CNS); compounds in these classes are distinct in that they have to cross the blood-brain barrier [Ghose, et al. J. Comb. Chem. 1, 55-68 (1999)].

In another embodiment, the physicochemical property is the total number of peripheral functionalities per molecule. In another embodiment, the physicochemical property is the total number of heavy atoms present in peripheral functionalities. Heavy atoms are atoms heavier than carbon and include, for example, N, O, S, P, F, Cl, Br, and I. These molecular parameters are of interest, because it is known that drugs in the CMC contain an average of four peripheral functionalities per drug molecule, and the average ratio of the number of heavy atoms present in peripheral functionalities to the number of peripheral functionalities is two [Bemis & Murcko J. Med. Chem. 42 5095-5099 (1999)]. To illustrate, the antimalarial agent quinine will be used herein; the structure of which is:

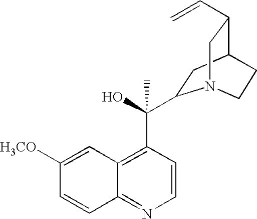

The molecular shape of quinine is defined by two ring systems separated by a linker. Furthermore, the total number of peripheral functionalities present for quinine is three, i.e., the methoxy group, the hydroxy group and the ethylene group. The total number of heavy atoms occurring in the peripheral functionalities is two, the oxygen belonging to the methoxy group on the quinoline and the oxygen belonging to the hydroxy group on the linker. Thus this particular molecule has a ratio of 0.66 for heavy atoms present in peripheral functionalities to the total number of peripheral functionalities, which is lower than the average.

In another embodiment, the physicochemical property is the logP. The logP is a measure of the overall lipophilicity of a compound such as a drug molecule, where P is the partition coefficient of the drug present as a neutral molecule, or in other words, where P is the ratio of the concentration of the drug in an organic solvent immiscible with an aqueous solvent to the concentration of the drug in the aqueous solvent.

In another embodiment, the physicochemical property is a calculated logP. LogP can be calculated a number of ways, such as through use of, for example, the method of Hansch and Leo (Hansch & Leo, Substituent Constants for Correlation Analysis in Chemistry and Biology, Wiley, New York, 1979, 18-43) as implemented by the Pomona College Medicinal Chemistry Project MedChem Software distributed by Biobyte (Leo, Chem. Rev. 93, 1281-1306 (1993); Leo, Chem. and Pharm. Bull., 43, 512 (1995)), the Moriguchi algorithm (Moriguchi et al., Chem. and Pharm. Bull, 40, 127-130 (1992); Moriguchi et al., Chem. and Pharm. Bull., 42, 976-978 (1994)), the Moriguchi algorithm as particularly implemented by Lipinski (Lipinski et al. Advanced Drug Discovery Reviews 23, 3-25 (1997)); the Suzuki-Kudo method (Suzuki & Kudo, J. Comput.-Aided Mol. Design, 4, 155 (1990); Suzuki, J. Comput.-Aided Mol. Design, 5, 149 (1991)); the method of Rekker (Rekker et al., Quant. Struct.-Act. Relat., 12, 152 (1993)); the method of Ghose and Crippen (Ghose & Crippen, J. Comput. Chem. 9 80 (1988); Ghose & Crippen, J. Comput. Chem. 7 565-577 (1986); Ghose, et al. J. Phys. Chem. A 102, 3762-3772 (1998)); the KOWWIN program (Meylan, & Howard, J. Pharm. Sci. 84, 83-92 (1995)); or the ACD/LogP program from Advanced Chemistry Development (online calculations at www.acdlabs.com/ilab).

In another embodiment, the physicochemical property is an experimental logP. LogP can be experimentally measured by use of the filter probe method (Tomlinson, J. Pharm. Sci. 71 602-604 (1982)), the shake flask method, or reverse-phase HPLC. Alternatively, pKₐ and logP can be measured simultaneously by use of potentiometric titration. The value of the partition coefficient can change depending upon the particular solvents used. Organic solvents including 1-octanol, chloroform, propylene glycol dipelargonate (PGDP), cyclohexane, alkanes, and phospholipids have been used to determine logP experimentally. LogP measurements taken in these solvents show differences that can be ascribed to different hydrogen bonding properties of the solvents. For example, 1-octanol is amphiprotic; chloroform is a hydrogen bond donor, and propylene glycol is a hydrogen bond acceptor. Alkanes are inert to hydrogen bonding. In another embodiment, the physicochemical property is the difference between experimental logP as measured in two different solvents. Interestingly, differences in experimental logP values, i.e., delta logPs, have been found to correspond with certain pharmaceutical activities. For example, logP(_{octanol-water})-logP(_{PDPG-water}) correlates with cardioselectivity of oxypropanolamines (Leahy, et al. in QSAR: Rational Approaches to the Design of Bioactive Compounds, Silipo & Vittoria, eds., Elsevier, Amsterdam, 1991 p. 75-82), and logP(_{octanol-water})-logP(_{cyclohexane-water}) inversely correlates with Log(Cbrain/Cblood) for a series of H2-receptor histamine antagonists (Ganellin, in QSAR: Rational Approaches to the Design of Bioactive Compounds, Silipo & Vittoria, eds., Elsevier, Amsterdam, 1991, p. 103-110).

In another embodiment of the method, the physicochemical property of the ligand candidates is at least one characteristic that contributes to the oral availability of drug molecules. In yet another embodiment, the physicochemical property is the number of hydrogen bond donors. In still another embodiment, the physicochemical property is the number of hydrogen bond acceptors. In another embodiment, the physicochemical property is molecular weight of the ligand candidate. In another embodiment, the physicochemical property is the clogP, where P is the calculated octanol/water partition coefficient. For optimal oral availability, the molecular weight of the compound generally should be 500 Da or less. Additionally, the number of hydrogen bond donors, i.e. —OH and —NH—, should be 5 or fewer and total number of hydrogen bond acceptors should be 10 or fewer. Finally, the clogP, should be either 5 or less as calculated according to Hansch-Leo, or 4.15 or less as calculated according to Moriguchi, to increase the chance of oral availability. The set of features including molecular weight, number of hydrogen bond donors, number of hydrogen bond acceptors, and calculated logP is referred to as Lipinski's rules or Rules of Five, derived from an analysis of molecules in the World Drug Index (WDI) [Lipinski et al. Advanced Drug Discovery Reviews 23, 3-25 (1997); Walters et al., Current Opinion in Chemical Biology 3, 384-387 (1999) review].

In the methods of the invention, each of the forgoing properties can be used either alone, in combination with each other, or in combination with other physicochemical properties described herein. For example, the compounds may be simultaneously categorized as to whether they meet the criteria of at least one alcohol and a total of five or fewer hydrogen bond donors. Such an analysis can be used to determine the proportion of drug-like molecules contained among molecules tethering to a particular cysteine-containing protein or collection of cysteine-containing proteins.

In another aspect, the invention is directed to a method for characterizing a molecular interaction site on a protein, the method comprising:

a) contacting a polypeptide comprising a thiol or a masked thiol at a site of interest with a library of ligand candidates wherein each ligand candidate comprises a disulfide group that is capable of engaging in disulfide exchange to form a disulfide linkage between the polypeptide and the ligand candidate;

b) forming the disulfide linkage between the polypeptide and a plurality of the ligand candidates;

c) detecting the ligand candidates that formed the disulfide linkage;

d) identifying a physicochemical property of at least one of the ligand candidates that formed the disulfide linkage; and

e) calculating the statistical significance of the incidence of ligand candidates having the physicochemical property in the plurality;

wherein the physicochemical property is selected from the group consisting of molecular weight, LogP, number of hydrogen bond donors, and number of hydrogen bond acceptors.

Other molecular properties may also be used in the methods described herein. In another embodiment, the molecular property is the number of rotatable bonds in the ligand candidate. In another embodiment, the molecular property is the number of chiral centers in the ligand candidates. In another embodiment the molecular property is polar surface area.

In another embodiment, the molecular property is derived from a topological index. Topological indices are 2-dimensional descriptors of molecules based on graph theory concepts that differentiate molecules in terms of various properties, including their overall shapes. They include, for example, Molecular Shape Kappa Indices [L. B. Kier Quant. Struct.-Act. Relat. 8, 218 (1989)]; Graph-Theoretical Information Content Indicies; Balaban Indices [Balaban, Chem. Phys. Let. 89 399-404 (1982)]; Molecular Flexibility Index and Kier and Hall Chi Connectivity Indices [Hall and Kier Reviews in Computational Chemistry II; Lipkowitz, K. B. and Boyd, D. B., Eds., VCH Publishers: New York; 367-422 (1991)]; the Wiener Index [Wiener J. Chem. Phys. 69 17-20 (1947); Muller et al., J. Comput. Chem., 8, 170-173 (1987)]; the Zagreb Index [Bonchev, Information Theoretic Indices for Characterization of Chemical Structures, Research Studies Press: Letchworth, England (1983)]; and Electrotopological-state keys (E-state keys) [Kier and Hall Pharm. Res. 7 801-807(1990), L. B. Kier and L. H. Hall Molecular Structure Description: The Electrotopological State, Academic Press, San Diego, 1999]. Graph-Theoretical Information Content Indices allow molecules to be viewed as structures that can be partitioned into elements that are in some sense equivalent. Balaban Indices are a measure of the branching of a molecule. The Molecular Flexibility Index is a descriptor that takes into account the number of atoms of a molecule, the presence of rings, and the presence of branching. In a somewhat related vein, the Kier and Hall Connectivity Indices are series of numbers that describe a molecule by taking into account branching, presence of ring structures, and flexibility. The Wiener Index is the sum of the chemical bonds existing between all pairs of heavy atoms in a molecule. The Zagreb Index is the sum of the squares of the vertex valencies. The E-state keys are descriptors of the atoms and bonds of molecules using electronic and topological attributes; E-state keys have been found to correlate with NMR chemical shifts, which also depend upon electronics and sterics. Any of these descriptors can be used to define properties of compounds capable of tethering to a site of interest, or to distinguish compounds binding the site of interest from compounds that do not bind to the site of interest, or from the overall library.

Another approach for characterizing a molecular interaction site is comparing the ligands that bind to the site with known drugs in terms of a specific physicochemical property. For example, among known drugs, the average logP is 2.52, as calculated using the ALOGP method [Ghose, et al. J. Phys. Chem. A 102, 3762-3772 (1998)]. This property can also be described in terms of different ranges around the mean describing different percentages of known drugs [Ghose, et al. J. Comb. Chem. 1, 55-68 (1999)]. Eighty percent of known drugs fall within a logP range of −0.4-5.6, and 50% of known drugs fall within the range of 1.3-4.1. The logP of the bound ligands, particularly whether the value is above or below 2.52 provides information regarding the hydrophobicity of the site.

Size-related properties such as molar refractivity, molecular weight, and number of atoms may also be detailed in this manner. For molar refractivity, which is a property that is a combined measure of the size and polarizability of a molecule, the average among known drugs is 97, with 80% of known drugs falling between 40-130, and 50% of known drugs falling between 70-110. For molecular weight the average is 357, with 80% of known drugs falling between 160-480, and 50% falling between 230-390. For number of atoms, the average is 48; with 80% of known drugs falling between 20-77 and 50% falling between 30-55.

It is also of interest to examine the physicochemical properties of drugs as they pertain to particular therapeutic classes. Examples of particular therapeutic classes include antipsychotic, antidepressant, hypnotic, antiinflammatory, antihypertensive, antineoplastic, and anti-infective. Particular classes of drugs tend to show different average values for certain physicochemical properties, and furthermore show a narrower distribution of numerical ranges than the corresponding numerical ranges for those properties for all known drugs [Ghose, el al. J. Comb. Chem. 1, 55-68 (1999)]. For example, CNS drugs have to cross the blood-brain barrier, as mentioned above, and hence tend to be more lipophilic than drugs belonging to other therapeutic classes. The average calculated logP according to Ghose-Crippen method is 1.59 for antineoplastic drugs but is 4.10 for antipsychotic drugs.

Characteristics of bound compounds can be profiled more narrowly such as those compounds that bind to different types of targets, to specific targets, and to particular sites of interest thereon. One method involves the use of a chemoprint that summarizes the characteristics of the bound compounds. Such information can then be combined with what is known about frameworks, properties and functionalities of drugs and used in the drug development process. For example, the information can be incorporated into the design of a biased or focused library of molecules for screening against a target, potentially increasing the likelihood molecules binding the target will possess drug-like properties. These parameters are used to choose which compounds from a set of library members binding the target should be developed further, and provide clues as to how they might be developed.

The methods for characterizing molecular interaction sites on proteins were applied to targets including HIV integrase.

HIV Integrase Molecular Interaction Sites

HIV-1 integrase is a 288 amino acid protein composed of N-terminal, C-terminal, and central catalytic core domains (Johnson et al., 1986. Proc Natl Acad Sci USA 83: 7648; Khan et al., 1991. Nucleic Acids Res 19: 851; Engelman A, Craigie R. 1992. J Virol 66: 6361; van Gent DC, et al., 1992. Proc Natl Acad Sci, USA 89: 9598; Leavitt et al., 1993. J Biol Chem 268: 2113; Vincent et al., 1993. J Virol 67: 425). Three-dimensional structures of all three isolated domains have been determined. All three domains are required for each step of the integration reaction (i.e., 3′-processing and strand-transfer reactions (Bushman et al., 1993. PNAS USA 90: 3428). If presented with a DNA substrate that mimics the immediate product of a strand transfer reaction, fragments containing at least the catalytic core (amino acid residues 52-210) catalyze an apparent reversal of the integration reaction, referred to as “disintegration” (Chow et al., 1992. Science 255: 723). While the physiological significance of this activity is unknown, disintegration presents a useful avenue for the characterization of integrase activities in vitro, particularly for the isolated catalytic core domain. The central catalytic core domain has a conserved triad of acidic amino acid residues (i.e., the “D,D-35-E” motif) widely found in retroviral integrases, eukaryotic retrotransposons, and various bacterial transposable elements. Each conserved acidic residue of the D,D-35-E motif is required for each step of the integration reaction (Engelman et al., supra; van Gent et al., supra; Leavitt et al., supra). The amino-terminal (residues 1-51) or “HHCC domain” has two histidines and two cysteines that are part of a zinc-binding motif (Burke et al., 1992. J Biol Chem 267: 9639; Bushman et al., 1993. Proc Natl Acad Sci USA 90: 3428; Zheng et al., 1996. Proc Natl Acad Sci USA 93: 13659; Yang et al., 1999. Journal of Virology 73: 1809) that is conserved among retroviral integrase proteins and yeast retrotransposons. This domain has been implicated in viral DNA binding (Ellison and Brown 1994. Proc Natl Acad Sci USA 91: 7316; Vink et al., 1994. Nucleic Acids Res 22: 4103; Donzella et al., 1996. Journal of Virology 70: 3909; van den Ent et al., 1999. Journal of Virology 73: 3176) and integrase oligomerization (Zheng et al., 1996, supra; Lee et al., 1997. Biochemistry 36: 173; Yang F, Roth M J. 2001. J. Virol. 75: 9561). The carboxy-terminal domain is the least conserved of the three integrase domains and includes at least amino acids 220-288 (Chen et al., 2000. Proc Natl Acad Sci USA 97: 8233). Although the carboxy-terminal domain has also been implicated in integrase oligomerization and viral DNA binding (Brown P O. 1997. Integration. In Retroviruses, ed. J M Coffin, S H Hughes, H E Varmus, pp. 161. Cold Spring Harbor: Cold Spring Harbor), mutagenesis experiments have shown that the domain binds DNA in a non-specific manner (Gerton J L, Brown P O. 1997. Journal of Biological Chemistry 272: 25809; Engelman et al., 1994. J. Virol 68: 5911; Lutzke et al., 1994. Nucleic Acids Res 22: 4125), suggesting involvement in host DNA binding. Structures of the individual N-terminal (Cai et al., 1997. Nature Structural 567; Cai et al., 1998. Protein Sci 7: 2669) and C-terminal domains (Eijkelenboom et al., 1995. Nat Struct Biol 2: 807; Lodi et al., 1995. Biochemistry 34: 9826; et al., 1999. Proteins 36: 556) have been determined by NMR. The cloning and mutagenesis of HIV integrase, and expression of the HIV integrase mutant proteins are described in Example 1.

Molecular interaction sites were characterized on HIV integrase by methods described herein. A number of cysteine mutants were screened against compounds in a ligand candidate library; each of the mutants could access at least the active site, cacodylate binding site, or the flexible loop region. Example 1 describes the protocols used to clone HIV integrase and cysteine mutants thereof. Each of the following cysteine mutants were present in the context of a polypeptide comprising residues 52-210 of SEQ ID. NO. 1 having substitutions comprising: C56S, C65A, C130A, W131D, and F185K. Substitutions to cysteine of residues within the active site of the catalytic core that were examined include D64; T66; H67; D116; I151; E152; N155; K156; and K159. Substitutions to cysteine within the cacodylate site that were examined include E92 and N120. Substitutions to cysteine within the flexible loop that were examined include H114, Q148, N144 and Q62. Numbering for the aforementioned residues is relative to the amino acid sequence of SEQ ID NO:1.

Each of the aforementioned mutants was contacted with libraries of ligand candidates, and the ligand candidates that formed the reversible covalent bond to the cysteine mutants were identified. The ligand candidates were also assessed for the presence or absence of a particular characteristic. The prevalence of molecules with the characteristic among the hits was compared with the prevalence of molecules with the characteristic among the total library, and the significance of any difference was measured. In some cases, referred to generally as enrichment, the incidence of molecules with a particular characteristic is greater in the population forming a protein-ligand conjugate with a particular cysteine mutant. Conversely, in other cases, referred to generally as depletion, the incidence of molecules with a particular characteristic is lower in the population forming a protein-ligand conjugate with a particular cysteine mutant than in the total library. Importantly, the significance of the enrichments and depletions, which is related to the number of compounds with the characteristic that were screened, is assessed. An illustrative protocol for determination of statistical significance of enrichments and depletions is given in Example 3.

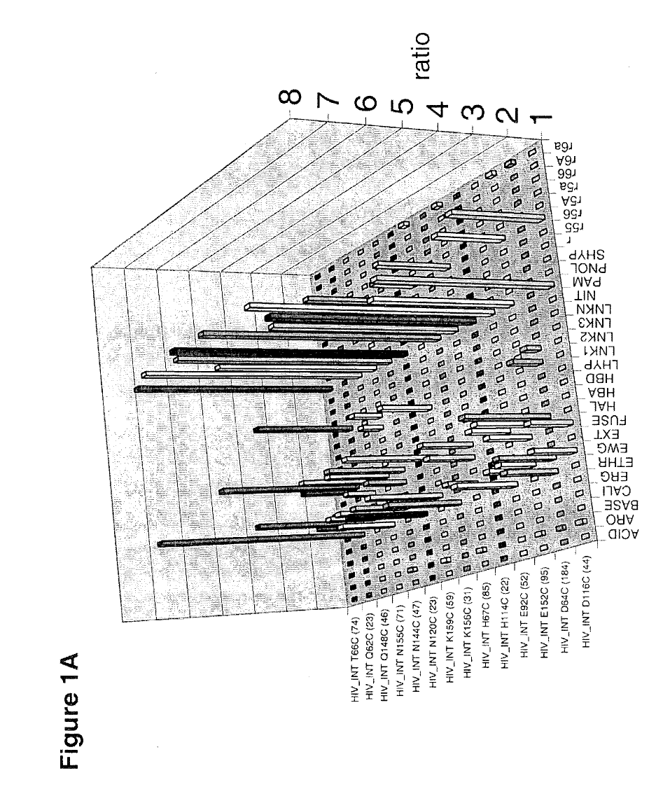

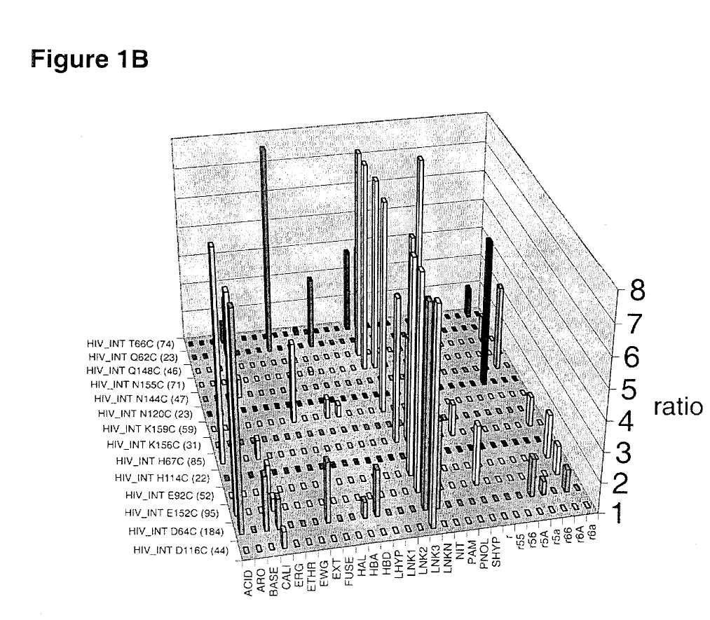

The significant enrichments and significant depletions for the screen are shown in FIG. 1A and FIG. 1B, respectively. Shown for each of the mutants tested are enrichments and depletions of ligand candidate characteristics having a 99% or greater chance of being statistically significant. Information may be obtained from significant enrichments, significant depletions, or both significant enrichments and significant depletions, as discussed below. The number of hits that were obtained and used in the analysis for each mutant is shown in parentheses to the right of that mutant. Preferably, conclusions will be based at least in part on significant enrichments or significant depletions resulting from a sizeable number of hits, at least greater than about 10. In the figure, descriptors are used to denote the ligand candidate chemical features under study. These descriptors are defined in Table 1.

TABLE 1

| Descriptor | Chemical Feature |
|---|---|
| ACID | acid group |
| ARO | aromatic group |
| BASE | base group |
| CALI | cyclic aliphatic system |
| ERG | electron-releasing group |
| ETHR | Ether |
| EWG | electron-withdrawing group |
| EXT | extended structure (long & flexible) |
| FUSE | fused ring system |
| HAL | Aryl halide |
| HBA | hydrogen bond acceptor |
| HBD | hydrogen bond donor |
| LHYP | large hydrophobic substituent |
| LNK1 | two rings separated by 1 bond |
| LNK2 | two rings separated by 2 bonds |
| LNK3 | two rings separated by 3 bonds |
| LNKN | two rings separated by 4 or more bonds |
| NIT | nitro group |
| PAM | primary amine or amine |
| PNOL | Phenol |
| SHYP | small hydrophobic substituents |
| R | Ring |
| r55 | 55 fused ring system |
| r56 | 56 fused ring system |
| r5A | 5-atom aliphatic ring |
| r5a | 5-atom aromatic ring |
| r66 | 66 fused ring system |
| r6A | 6-atom aliphatic ring |
| r6a | 6-atom aromatic ring |

Generally speaking, physicochemical properties relating to chemical features, such those as delineated in Table 1, can be detected manually or using a computer program. Commercially available options include the MDL MOL file substructure query capability found in the ISIS family of products, the SMARTS chemical query language found in products from Daylight Chemical Information Systems, and the modified SMARTs language found in the OELib and OEChem packages from Open Eye Scientific Software. An equivalent chemical feature detection algorithm can also be used in a custom-written program using a language such as C, C++, or Python.

For most of the mutants of HIV integrase examined, there is a large significant enrichment of primary amines (PAM) among the hits, as can be seen in FIG. 1A.

Among mutants showing this enrichment, ratios range from 5-11. This enrichment is target-specific, in that it was not observed among other targets examined, for example, see IL-1R in Example 4.

Likewise, a large significant depletion of two rings connected by three bonds is also observed at the majority of integrase residues studied, as can be seen in FIG. 1B. Among mutants displaying this depletion, ratios range from 4-15. Although depletions in this characteristic are observed to some extent with other targets, such as IL-1R in Example 4, the effect is not nearly as strong nor at as many residues. It would thus appear that compounds comprising two rings connected by three bonds can be used as negative controls for similar compounds showing binding to HIV integrase.

Interestingly, information derived from significant enrichments and significant depletions may be used together in order to characterize a site of interest. For example, the H67C mutant shows significant enrichment for 56 fused ring systems (3-fold) but also shows a significant depletion for 6-atom aliphatic rings (2-fold). This pattern of enrichments and depletions, taken together, suggests a specific interaction site proximal to this position for 56 fused ring systems comprising a 6-membered aromatic or partially unsaturated ring. Similar enrichment and depletion ratios for these groups are observed for E152C, which is also a part of the active site.

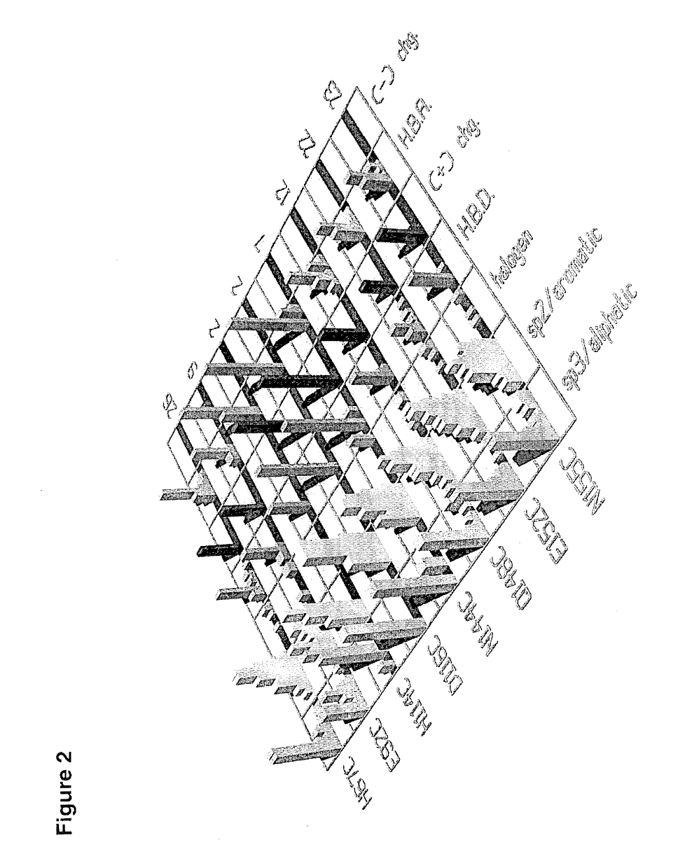

Unlike methods involving examination of solely noncovalent interactions, positional information about binding of the ligand candidates to a molecular interaction site on a target may be obtained in a number of ways using the methods herein. The residue to which the fragment is conjugating is known. Furthermore, the distance between a given chemical feature and the second chemically reactive group on the ligand candidate is known. Because some molecules are flexible, the distance may be measured as the number of intervening bonds between the chemical feature and the second chemically reactive group. Such an analysis, called a linear chemoprint, is shown in FIG. 2 for ligands conjugating to various residues of HIV integrase. Distance from the tether position for each chemical feature increases from left to right in the figure, beginning at three bonds away from the second chemically reactive group, a thiol in this case. It can be seen that different mutants have different preferences for the location of a given atom type or functionality relative to the location of the second chemical group at the tether position on the ligand candidate. For example, whereas the mutants N155C and E152C have a similar number of hits (23 and 22, respectively), they exhibit different preferences for the location of hydrogen bond donors on the ligand candidates. Ligand candidates conjugating the N155C mutant have hydrogen bond donors predominately five bonds away from the ligand candidate thiol, with a small number seven bonds away from the ligand candidate thiol, but ligand candidates conjugating the E152C mutant have hydrogen bond donors in many different positions with a slight preference for those five bonds away from the ligand candidate thiol. Both mutants are part of the active site, however, the N155C mutant is more sensitive to the location of the hydrogen bond donor relative to the ligand candidate thiol group.

Another way to determine information about where the compound is binding on the protein is to vary the distance between the second chemically reactive group of the ligand candidate and other chemical features of the ligand candidate. The effect of the change in distance can be measured in two ways, a β-ME₅₀ and the fraction of cysteine mutant conjugated under a given concentration of β-ME. The β-ME₅₀ is the concentration of β-mercaptoethanol under which 50% of the target conjugates to the ligand candidate. This value is an estimate of the strength of an interaction of a given ligand candidate with a given cysteine mutant; the higher the β-ME₅₀ at a given cysteine mutant, the stronger the interaction between the ligand candidate and the mutant. Examples of these types of results are shown in Table 2. As can be seen in Table 2, for several scaffolds, the distance between the ligand candidate thiol group and each of the remaining chemical features defining the ligand candidate was increased by one bond length, corresponding to an additional methylene group added in the vicinity of the ligand candidate thiol group.

TABLE 2

| | | Mutant/ | |
|---|---|---|---|
| Scaffold | n | β-ME-50 (mM) | |
| 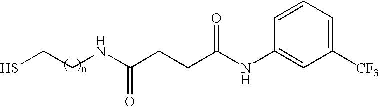 | 1 | Q148C | 1.17 |
| | 2 | Q148C | 0.212 |
| 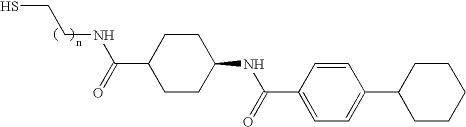 | 1 | H67C | 1.02 |
| | 2 | H67C | 0.614 |
| 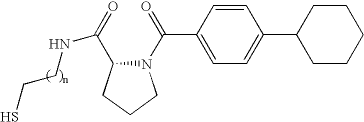 | 1 | N155C | 1.02 |
| | 2 | N155C | 2.43 |
| 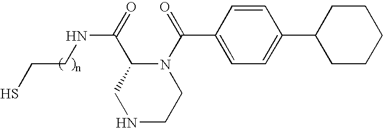 | 1 | N155C | 1.93 |
| | 2 | N155C | 3.19 |
| 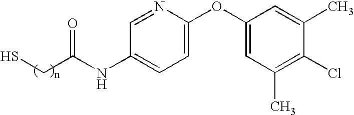 | 1 | Q62C | 6.4 |
| | 2 | Q62C | 4.3 |

The additional distance between the thiol and the remaining chemical features had differing effects upon the β-ME₅₀ depending upon the scaffold. For example, in the first scaffold shown in Table 2, the addition of only one methylene decreased the β-ME₅₀ by a factor of about 5. The second scaffold also shows a decrease in β-ME₅₀ upon addition of an extra methylene, but for conjugation to the H67C residue. On the other hand, the addition of a methylene to the third scaffold increased the β-ME₅₀ for conjugation to the N155C residue by a factor of 2.5. The fourth scaffold on Table 2, having a similar structure to the third scaffold on Table 2 shows a similar linker length dependence of conjugating to the same residue (N155C). The fifth scaffold shows a decrease in the β-ME₅₀ for conjugation to Q62C upon addition of the extra methylene.

As mentioned above, similar information on the effect of the distance of a chemical feature from the second chemically reactive group on binding to the polypeptide can be obtained using the fraction conjugated as a readout, shown in Table 3.

TABLE 3

| | | |
|---|---|---|
| | | Mutant/Fraction |
| Scaffold | n | conjugated |

| | | | |
|---|---|---|---|
| 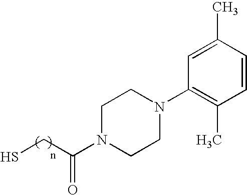 | 1 2 | K159C K159C | 0.71 0.28 |
| 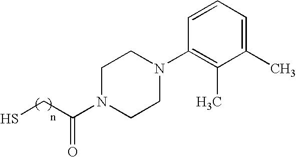 | 1 2 | K159C K159C | 0.66 0.30 |
| 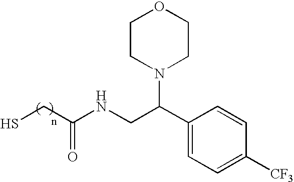 | 1 2 | N155C N155C | 0.23 <0.1 |
| 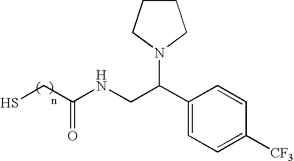 | 1 2 | N155C N155C | 0.33 <0.1 |
| 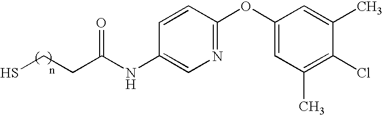 | 1 2 | Q62C D64C Q62C D64C | 0.34 0.60 0.86 0.54 |
| 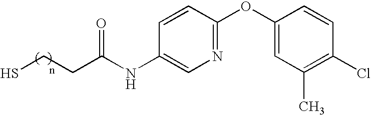 | 1 2 | Q62C D64C Q62C D64C | 0.27 0.30 0.82 0.57 |

As can be seen in Table 3, the first two scaffolds show a similar effect of increased distance between the thiol group and the remainder of the scaffold, that is, a decrease in the fraction conjugated by more than a factor of 2 to residue K159. The third scaffold and the fourth scaffold, which are structurally related each show a decrease in the fraction conjugated to residue N155C upon the increase in the distance between the thiol and the remainder of the scaffold. The fifth scaffold was tested against two mutants, Q62C and D64C. As it can be seen, the extra methylene increased the fraction conjugated for the Q62C mutant by more than a factor of 2, whereas there was only a negligible effect for the D64C mutant with the same compounds. The Q62C and D64C mutants were also tested with the sixth scaffold on Table 3. The only difference between the fifth and the sixth scaffolds is the presence of an additional methyl group on the terminal phenyl ring of the fifth scaffold on Table 3. For the sixth scaffold the effect of the extra methylene between the thiol and the remainder of the scaffold had a similar effect for both cysteine mutants tested, i.e. it increased the fraction bound by about a factor of 3 for Q62C and by about a factor of 2 for D64C. It is possible that the extra methylene on the fifth scaffold enables the additional methyl of this scaffold to make a favorable hydrophobic contact on HIV integrase from Q62C. Such a hydrophobic contact for the fifth scaffold may be already accessible in the presence of only a single methylene for D64C; the removal of the methyl from the compound (see Table 3, sixth scaffold, n=1) decreases fraction bound at this position, also by a factor of 2. Increasing by one methylene in the context of the sixth scaffold restores the fraction bound of D64C to the same level observed for the fifth scaffold with one methylene group.

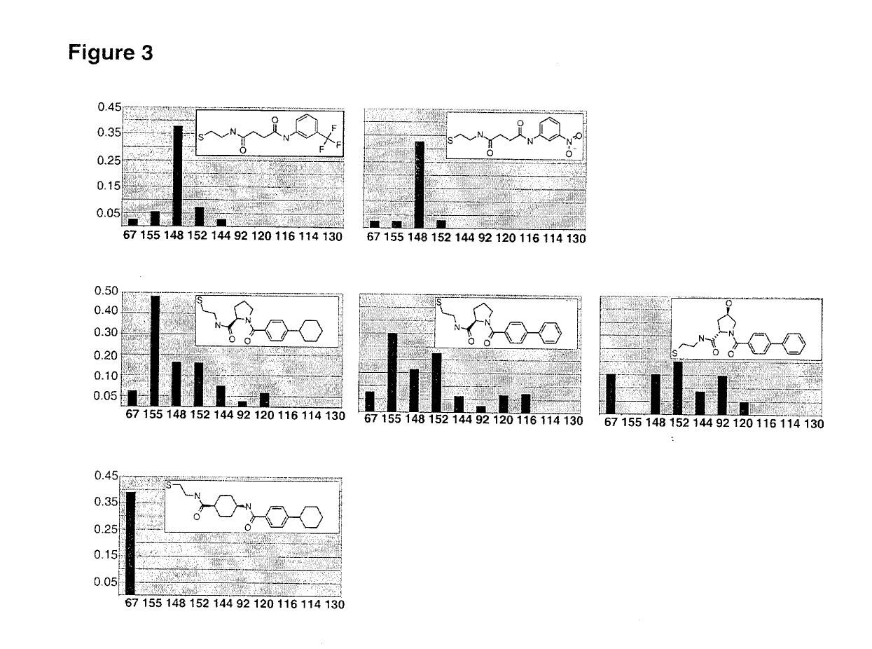

In another variation of the methods described herein, the protein is individually contacted with members of the library of ligand candidates. The measurement of interactions between protein and individual ligand candidates is preferably performed with purified compounds that already have been shown to tether to the protein when present in a pool of compounds. One reason to examine tethering of individual compounds to the protein is that the fraction of the protein that becomes covalently attached to the compound to form the protein-compound conjugate, i.e. the strength of the hit, may be obtained. This hit strength may be used as a threshold value. For example, in a given experiment, it may be decided that only compounds capable of conjugating at least 5% of the total amount of the polypeptide are to be counted as having formed the reversible covalent bond. The strength of a hit can also be compared across a set of cysteine mutants. This type of an analysis is referred to a specificity profile. Shown in FIG. 3 is a specificity profile for several compounds that conjugate to at least one cysteine mutant of HIV integrase. The fraction of each of the H67C, N155C, Q148C, E152C, N144C, E92C, N120C, D116C, H114C and C130A mutants forming a conjugate with the compound was measured. It can be readily seen that some of the compounds have good specificity against the plurality of mutants (top panel), with reaction at a small subset of the mutants examined, and a large difference between the extent of conjugate formation with the preferred mutant (Q148C) than the other mutants. Some of the compounds exhibit a weak degree of specificity (center panel), showing reactivity with many of the mutants, and a small extent of difference between the preferred mutants over the nonpreferred mutants. Finally, one compound shows a unique reaction with the H67C mutant. Furthermore, the compounds shown have structural similarities to one another, and small changes in the compounds have an effect on the specificity of conjugation observed. For example, if the compounds in the first column, row 2 and first column, row 3 are compared, replacement of a pyrrolidine ring with a cyclohexylamino ring switches the preferred site of conjugation from N155C to H67C and sharpens the specificity of the compound for the H67C position. When a specificity profile is performed for a substantial number of compounds, molecular interaction sites on the protein may be characterized in terms of location of hits, chemical features of the hits, and strength of the hits.

The foregoing discussion concerned the characterization of different types of molecular interaction sites on an enzyme; the method can also be used to characterize sites that are important for protein-protein binding. Example 2 describes the cloning and mutagenesis of one such target, interleukin-1 receptor. Example 4 demonstrates this type of characterization for human intereleukin-1 receptor, type 1 (IL-1R1). In addition, the invention is further illustrated by the following non-limiting Examples.

### EXAMPLES

### EXAMPLE 1

Cloning and Mutagenesis of Human Immunodeficiency Virus Integrase (HIV IN)

Cloning of HIV IN

Numbering of the wild type and mutant HIV-1 integrase residues follows the convention of the first amino acid residue of the mature protein being residue number 1, and the HIV-1 integrase catalytic core domain being comprised of residues 52-210 [Leavitt, A. D., et al., J Biol Chem 268: 2113-2119 (1993)].

A plasmid construct, pT7-7 HT-INₜₑₜᵣₐ, encoding the HIV integrase core domain (residues 50-212), having an N-terminal 6× histidine tag and thrombin cleavable linker, and C56S, W131D, F139D, and F185K mutations in the pT7-7 (Novagen) vector background [Chen, J. C.-H., et al., Proc. Natl. Acad. Sci. U.S.A. 97: 8233-8238 (2000)] was obtained from Dr. Andy Leavitt at UCSF. Upon comparison of the crystal structure of this core domain variant [Chen, J. C.-H., et al., Proc. Natl. Acad. Sci. U.S.A. 97: 8233-8238 (2000)] to other integrase core structures, it was noted that the F139D mutation, designed to increase solubility of the protein, caused a rotation of the side chain that transmitted a distortion to the catalytically important Asp116. The mutation was therefore reverted to the wild-type phenylalanine residue by Quickchange mutagenesis (Stratagene), following manufacturer's instructions and using SEQ ID NO:2 and SEQ ID NO:3.

| | |
|---|---|
| D139F1-int | |
| GTATCAAACAGGAATTCGGTATCCCGTACAAC | SEQ ID NO:2 |
| D139F2-int | |
| GTTGTACGGGATACCGAATTCCTGTTTGATACC | SEQ ID NO:3 |

This generated pT7-7 HT-INₜᵣᵢ, encoding the triple mutant (C56S, W131D, F185K) of the integrase core, SEQ ID NO: 4.

| | |
|---|---|
| 52 | GQVDSSPGIW QLDCTHLEGK VILVAVHVAS GYIEAEVIPA ETGQETAYFL LKLAGRWPVK |
| 112 | TIHTDNGSNF TGATVRAACD WAGIKQEFGI PYNPQSQGVV ESMNKELKKI IGQVRDQAEH |
| 172 | LKTAVQMAVF IHNKKRKGGI GGYSAGERIV DIIATDIQT |

In preparation for making cysteine mutations at tethering sites, the two wild-type cysteines, (C130 and C65) were replaced by alanine residues and the DNA encoding the His-tagged INₜᵣᵢ core domain transferred into the pRSET A vector, containing an F1 origin of replication that allows preparation of single-stranded plasmid DNA, and thus mutagenesis by the Kunkel method [Kunkel, T. A., et al., Methods Enzymol. 204: 125-139 (1991)]. Replacement of C130 by alanine was accomplished by cassette mutagenesis, using the double stranded cassette composed of SEQ ID NO:5 and SEQ ID NO:6. The cassette, containing the appropriate overhangs at each end, was ligated into pT7-7 HT-INₜᵣᵢ digested with BsiWI and EcoRI.

| | |
|---|---|
| C130A cassette 1 | |
| GTACGTGCTGCAGCCGACTGGGCTGGTATCAAACAGG | SEQ ID NO:5 |
| C130A cassette 2 | |
| GAATTCCTGTTTGATACCAGCCCAGTCGGCTGCAGCAC | SEQ ID NO:6 |

The C65A mutation was carried out independently by Quickchange mutagenesis on pT7-7 HT-INₜᵣᵢ using SEQ ID NO:7 and SEQ ID NO:8.

| | |
|---|---|
| C65A1-int | |
| ATCTGGCAACTGGACGCGACTCACCTCGAGGGT | SEQ ID NO:7 |
| C65A2-int | |
| ACCCTCGAGGTGAGTCGCGTCCAGTTGCCAGAT | SEQ ID NO:8 |

The DNA encoding HT-C130A integrase core domain was subcloned into the pRSET A vector by PCR cloning. SEQ ID NO:9 and SEQ ID NO:10 were used as PCR primers, and the resulting amplified product was digested with NdeI and Hind III, and ligated into pRSET A that had been digested with the same enzymes, to generate pRSET-HT-C130A-INₜᵣᵢ.

| | |
|---|---|
| C130_rsetF | |
| GGAGATATACATATGCACCACCATCACC | SEQ ID NO:9 |
| C130_rsetR | |
| ATCATCGATGATAAGCTTCCTAGGTCTGG | SEQ ID NO:10 |

A BamHI fragment of pT7-7 HT-C65A-INₜᵣᵢ containing the C65A mutation was ligated into pRSET-HT-C130A-INₜᵣᵢ, to generate pRSET-HT-INₜₑₘₚₗₐₜₑ. This plasmid served as a template for further Kunkel mutagenesis to introduce cysteine substitutions at positions chosen for tethering. The primer AATACGACTCACTATAG (SEQ ID NO:11) was used for sequencing.

Mutagenic Oligonucleotides

SEQ ID NO: 12

| | |
|---|---|
| Q62C | GTGAGTCGCGTCCAGGCACCAGATACCCGG |

SEQ ID NO:13

| | |
|---|---|
| D64C | CTCGAGGTGAGTCGCGCACAGTTGCCAGATAC |

SEQ ID NO: 14

| | |
|---|---|
| T66C | CTTTACCCTCGAGGTGACACGCGTCCAGTTGCC |

SEQ ID NO: 15

| | |
|---|---|
| H67C | GGATAACTTTACCCTCGAGGCAAGTCGCGTCCAGTTG |

SEQ ID NO: 16

| | |
|---|---|
| L68C | AACTTTACCCTCGCAGTGAGTCGCGTCCA |

SEQ ID NO: 17

| | |
|---|---|
| K71C | GCAACCAGGATAACGCAACCCTCGAGGTG |

SEQ ID NO: 18

| | |
|---|---|
| E92C | CAGTTTCCTGACCAGTGCAGGCCGGGATAACTTC |

SEQ ID NO:19

| | |
|---|---|
| H114C | GGATCCGTTGTCAGTGCAGATGGTTTTAACCGGC |

SEQ ID NO:20

| | |
|---|---|
| D116C | GTTGGATCCGTTGCAAGTGTGGATGGTTTTAACCG |

SEQ ID NO:21

| | |
|---|---|
| N120C | CGGTAGCACCAGTGAAGCAGGATCCGTTGTCAGTG |

SEQ ID NO:22

| | |
|---|---|
| N144C | CACCCTGAGACTGCGGGCAGTACGGGATACCGA |

SEQ ID NO:23

| | |
|---|---|
| Q148C | CATAGATTCAACAACACCGCAAGACTGCGGGTTGT |

SEQ ID NO:24

| | |
|---|---|
| 1151C | GCTCTTTGTTCATAGATTCGCAAACACCCTGAGA |

SEQ ID NO:25

| | |
|---|---|
| E152C | GCTCTTTGTTCATAGAGCAAACAACACCCTGAGA |

SEQ ID NO:26

| | |
|---|---|
| N155C | CCGATGATTTTTTTGAGCTCTTTGCACATAGATTCAACAAC |

SEQ ID NO:27

| | |
|---|---|
| K156C | CCGATGATTTTTTTGAGCTCGCAGTTCATAGATTC |

SEQ ID NO:28

| | |
|---|---|
| K159C | CCTGACCGATGATTTTGCAGAGCTCTTTGTTCAT |

SEQ ID NO:29

| | |
|---|---|
| G163C | CCTGATCACGAACCTGGCAGATGATTTTTTTG |

SEQ ID NO:30

| | |
|---|---|
| Q168C | GGTTTTCAGGTGTTCAGCGCAATCACGAACCTGA |

SEQ ID NO:31

| | |
|---|---|
| T174C | GCCATCTGAACCGCGCATTTCAGGTGTTCAGCC |

Expression of IN Cysteine Mutants

pT7-7 and pRSET integrase core domain expression plasmids were transformed into BL21 star E. coli (Invitrogen) by standard methods, and a single colony from the resulting plate was used to inoculate 250 mL of 2×YT broth containing 100 μg/mL ampicillin. Following overnight growth at 37° C., the cells were harvested by centrifugation at 4K rpm and resuspended in 100 mL 2YT/amp. 40 mL of the washed cells was used to inoculate 1.5 L of the same media, and after growth at 37° C. to an OD at 600 nm of between 0.5 and 0.8, the culture was moved to 22° C. and allowed to cool. IPTG was added to a final concentration of 0.1 mM and expression continued 17-19 h at 22° C. Cells were harvested by centrifugation at 4K rpm. Cell pellets were resuspended in 100 mL Wash 5 buffer (Wash 5: 20 mM Tris-HCl, 1 M MgCl₂, 5 mM imidazole, 5 mM β-mercaptoethanol, pH 7.4) and lysis was accomplished by sonication for 1 minute, repeated a total of 3 times with 2 minutes rest between. Cell debris was removed by centrifugation at 14K rpm followed by filtration. Integrase core domain was purified by affinity chromatography on Ni-NTA superflow resin (Qiagen) at 4° C. After loading the cell lysate, the column was washed with Wash 40 buffer (Wash 40: 20 mM Tris-HCl, 0.5 M NaCl, 40 mM imidazole, 5 mM β-mercaptoethanol, pH 7.4) and His-tagged IN core domain eluted with E400 buffer (E400: 20 mM Tris-HCl, 0.5 M NaCl. 400 mM imidazole, 5 mM β-mercaptoethanol). The purified enzyme was dialyzed versus 20 mM Tris, 0.5 M NaCl, 2.5 mM CaCl₂, 5 mM β-mercaptoethanol, pH 7.4 at 4° C., and aliquoted into 1.5 mL tubes. Biotinylated thrombin (Novagen) (2U thrombin/mg of protein) was added and the tubes rotated overnight at 4° C., followed by thrombin removal using streptavidin-agarose resin (Novagen) and separation of His-tagged protein and peptides from the cleaved material by passage through a second column of Ni-NTA sepharose fast-flow. Purified, cleaved integrase core domain was dialyzed against 20 mM Tris-HCl, 0.5 M NaCl, 3 mM DTT, and 5% glycerol, pH 7.4, and stored at −20° C. Protein concentrations were determined by absorbance at 280 nm after desalting on NAP-5 columns (Pharmacia), using ε₂₈₀^{1%}=(1.174), and molecular weights confirmed by ESI mass spectrometry (Finnigan).

### Example 2

Cloning and Mutagenesis of Human Interleukin-1 Receptor Type I (IL-1R1)

Cloning of Human IL-1 Receptor Type I

The IL-1 receptor has three regions: an N-terminal extracellular region, a transmembrane region, and a C-terminal cytoplasmic region. The extracellular region itself contains three immunoglobin-like C2-type domains. The constructs used here contain the two N-terminal domains of the extracellular region. Numbering of the wild type and mutant IL1R residues follows the convention of the first amino acid residue (L) of the mature protein being residue number 1 after processing of the signal sequence [Sims, J. E., et al., Proc. Natl. Acad. Sci. U.S.A. 86: 8946-8950 (1989)]. The sequence of the 2 domain protein is shown below as SEQ ID NO:32.

| | |
|---|---|
| 1 | LEADKCKERE EKIILVSSAN EIDVRPCPLN PNEHKGTITW YKDDSKTPVS TEQASRIHQH |
| 61 | KEKLWFVPAK VEDSGHYYCV VRNSSYCLRI KISAKFVENE PNLCYNAQAI FKQKLPVAGD |
| 121 | GGLVCPYMEF FKNENNELPK LQWYKDCKPL LLDNIHFSGV KDRLIVMNVA EKHRGNYTCH |
| 181 | ASYTYLGKQY PITRVIEFIT LEENK |

In brief, cysteine mutants were made in the context of a 2 domain receptor and a 2 domain receptor with a his tag. In addition, the constructs possessed a mutation at a glycosylation site, and one construct possessed a mutation at a glycosylation site in addition to a deletion at the C-terminal residue of the 2 domain region. The assembly of these constructs is described below.

The DNA sequence encoding human Interleukin-1 receptor (IL1R) was isolated by PCR from a HepG2 cDNA library (ATCC) using PCR primers (IL1RsigintFor 5′; IL1RintRev 5′) corresponding to the signal sequence and the end of the extracellular domain of the protein.

| | |
|---|---|
| IL1RsigintFor | |
| TTACTCAGACTTATTTGTTTCATAGCTCTA | SEQ ID NO:33 |
| IL1RintRev | |
| GAAATTAGTGACTGGATATATTAACTGGAT | SEQ ID NO:34 |

The appropriate sized band was isolated from an agarose gel and used as the template for a second round of PCR using oligos (IL1RsigFor; IL1R319Rev), which were designed to contain restriction endonuclease sites EcoRI and XhoI for subcloning into a pFBHT vector.

| | |
|---|---|
| IL1Rsig For | SEQ ID NO:35 |

CCGGAATTCATGAAAGTGTTACTCAGACTTATTTGTTTC

| | |
|---|---|
| 1L1R319 Rev | SEQ ID NO:36 |

CCGCTCGAGTCACTTCTGGAAATTAGTGACTGGATATATTAA

The pFBHT vector is modified from the original pFastBac1 (Gibco/BRL) by cloning the sequence for TEV protease followed by (His)₆ tag and a stop signal into the XhoI and HinDIII sites. The PCR product containing the IL1R sequence was cut with restriction endonucleases (41 μl PCR product, 2 μl each endonuclease, 5 μl appropriate 10× buffer; incubated at 37° C. for 90 minutes). The pFBHT vector was cut with restriction endonucleases (6 μg DNA, 4 μl each endonuclease, 10 μl appropriate 10× buffer, water to 100 μl; incubated at 37° C. for 2 hours; add 2 μl CIP and incubated at 37° C. for 45 minutes). The products of nuclease cleavage were isolated from an agarose gel (1% agarose, TBE buffer) and ligated together using T4 DNA ligase (200 ng pFBHT vector, 150 ng IL1R PCR product, 4 μl 5× ligase buffer [300 mM Tris pH 7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4° C. for 20 minutes, 25° C. for 10 minutes), and plated onto LB/agar plates containing 100 μg/ml ampicillin. After incubation at 37° C. overnight, single colonies were grown in 3 ml 2YT media for 18 hours. Cells were then isolated and double-stranded DNA extracted from the cells using a Qiagen DNA miniprep kit. A 2-domain version of IL1R was created by PCR using the 3-domain IL1R-FBHT clone as a template. PCR was performed using the primers IL1RsigFor (SEQ ID NO:35) corresponding to the signal sequence, in addition to one of the following two reverse primers. The reverse primers are IL1R2Drevstop-Xho, which corresponds to the end of the second extracellular domain of the protein with a stop signal, and IL1R2Drev-Xho, which corresponds to the end of the second extracellular domain of the protein without a stop signal to create a fusion with the TEV protease site and the His tag.

| | | |
|---|---|---|
| IL1R2Drevstop-Xho | CCGCTCGAGTCATCATTTGTTTTCCTCTAGAGTAATAAA | SEQ ID NO:37 |
| IL1R2Drev-Xho | CCCCTCGAGTCATTTGTTTTCCTCTAGAGTAATAAA | SEQ ID NO:38 |

The PCR primers contain restrictions sites (EcoRI at the 5′end and XhoI at the 3′ end), which were used to ligate the 2-domain version into the pFBHT vector. The PCR product containing the IL1R2D sequence was cut with restriction endonucleases (41 μl PCR product, 2 μl each endonuclease, 5 μl appropriate 10× buffer; incubated at 37° C. for 90 minutes). The products of nuclease cleavage were isolated from an agarose gel (1% agarose, TBE buffer) and ligated together using T4 DNA ligase (200 ng pFBHT vector, 150 ng IL1R2D PCR product, 4 μl 5× ligase buffer [300 mM Tris pH 7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4° C. for 20 minutes, 25° C. for 10 minutes), and plated onto LB/agar plates containing 100 μg/ml ampicillin. After incubation at 37° C. overnight, single colonies were grown in 3 ml 2YT media for 18 hours. Cells were then isolated and double-stranded DNA extracted from the cells using a Qiagen DNA miniprep kit.

Additionally, the two glycosylation sites within IL1R2D, N83 and N176, were each individually mutated to a histidine, in order to make a more homogeneous protein. Each of these single mutants were made in the context of the 2-domain protein without a his tag (sIL1Rd2-FB) and the 2-domain protein with a his tag (sIL1Rd2-FBHT). Mutation was accomplished by PCR using two sets of primers to make two fragments, followed by stitching together of the fragments using the outside primers IL1RsigFor (SEQ ID NO:35) and either IL1R2Drevstop-Xho (SEQ ID NO:37) or IL1R2Drev-Xho (SEQ ID NO:38) as described below. Brief descriptions of the 2-domain glycosylation mutants and their construction follow.

The construct for the N83H mutant without a his tag is referred to as sIL1R2D-N83H-FB, and it was created using IL1RsigFor (SEQ ID NO:35) and N83HR (SEQ ID NO:39) along with N83HF (SEQ ID NO:40), and IL1R2Drevstop-Xho (SEQ ID NO:37).

| | | |
|---|---|---|
| N83HR | GAGGCAGTAAGATGAATGTCTTACC | SEQ ID NO:39 |
| N83HF | CTATTGCGTCGTAAGACATTCATCTT | SEQ ID NO:40 |

The construct for the N83H mutant with a his tag is referred to as sIL1R2D-N83H-FBHT and was created using IL1RsigFor (SEQ ID NO:35), and N83HR (SEQ ID NO:39) along with N83HF (SEQ ID NO:40) and IL1R2Drev-Xho (SEQ ID NO:38). The construct for the N176H mutant without a his tag is referred to as sIL1R2D-N176H-FB and it was created using IL1RsigFor (SEQ ID NO:35), N176HR (SEQ ID NO:41), N176HF (SEQ ID NO:42), and IL1R2Drevstop-Xho (SEQ ID NO:37).

| | |
|---|---|
| N176HR | |
| ATGACAAGTATAGTGCCCTCTATGCTTTTCACG | SEQ ID NO:41 |
| N176HF | |
| GCTGAAAAGCATAGAGGGCACTATACTTGTCAT | SEQ ID NO:42 |

The construct for the N176H mutant with a his tag is referred to as sIL1R2D-N176H-FBHT and it was created using IL1RsigFor (SEQ ID NO:35), and N176HR (SEQ ID NO:41), along with N176HF (SEQ ID NO:42), and IL1R2Drev-Xho (SEQ ID NO:38). The PCR products were isolated from and agarose gel and PCR was used to sew the two fragments together using the IL1RsigFor (SEQ ID NO:35) and IL1R2Drevstop-Xho (SEQ ID NO:37) or IL1R2Drev-Xho primers (SEQ ID NO:38). The PCR products containing the IL1 R2D sequences mutated at the glycosylation site were cut with restriction endonucleases (41 μl PCR product, 2 μl each endonuclease, 5 μl appropriate 10× buffer; incubated at 37° C. for 90 minutes). The products of nuclease cleavage were isolated from an agarose gel (1% agarose, TBE buffer) and ligated together using T4 DNA ligase (200 ng pFBHT vector, 150 ng IL1R2D PCR product, 4 μl 5× ligase buffer [300 mM Tris pH 7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4° C. for 20 minutes, 25° C. for 10 minutes), and plated onto LB/agar plates containing 100 μg/ml ampicillin. After incubation at 37° C. overnight, single colonies were grown in 3 ml 2YT media for 18 hours. Cells were then isolated and double-stranded DNA extracted from the cells using a Qiagen DNA miniprep kit. The subsequent plasmids are referred to as sIL1R2D-N83H-FB or sIL1R2D-N83H-FBHT and as sIL1R2D-N176H-FB or as sIL1R2D-N176H-FBHT. Finally, an additional construct was made using the sIL1R2D-N83H-FB construct. The additional construct contains the 2-domain IL1R receptor without a his tag and with two mutations: a N83H glycosylation mutation and a deletion of the C-terminal residue (K205). This construct is named sIL1R2D2M-FB, and was made using the K205del oligonucleotide.

| | | |
|---|---|---|
| K205del | CTCGAGTCATCAGTTTTCCTCTAG | SEQ ID NO:43 |

Generation of IL-1R1 Cysteine Mutations

Site-directed mutants of IL1R2D were prepared by the single-stranded DNA method [modification of Kunkel, T. A., Proc. Natl. Acad. Sci. U.S.A. 82: 488-492 (1985)]. Oligonucleotides were designed to contain the desired mutations and 15-20 bases of flanking sequence.

The single-stranded form of the IL1R2D (sIL1R2D-FBHT, sIL1R2D-N176H-FB/FBHT, sIL1R2D-N83H-FB/FBHT, sIL1R2D2M-FB) plasmid was prepared by transformation of double-stranded plasmid into the CJ236 cell line (1 μl IL1R-FB double-stranded DNA, 2 μl 2×KCM salts, 7 μl water, 10 μl PEG-DMSO competent CJ236 cells; incubated at 4° C. for 20 minutes and 25° C. for 10 minutes; plated on LB/agar with 100 μg/ml ampicillin and incubated at 37° C. overnight). Single colonies of CJ236 cells were then grown in 50 ml 2YT media to midlog phase; 10 μl VCS helper phage (Stratagene) were then added and the mixture incubated at 37° C. overnight. Single-stranded DNA was isolated from the supernatant by precipitation of phage (⅕ volume 20% PEG 8000/2.5 M NaCl; centrifuge at 12K for 15 minutes.). Single-stranded DNA was then isolated from phage using Qiagen single-stranded DNA kit.

Site-directed mutagenesis was accomplished as follows. Oligonucleotides were dissolved to a concentration of 10 OD and phosphorylated on the 5′ end (2 μl oligonucleotide, 2 μl 10 mM ATP, 2 μl 10× Tris-magnesium chloride buffer, 1 μl 100 mM DTT, 10 μl water, 1 μl T4 PNK; incubate at 37° C. for 45 minutes). Phosphorylated oligonucleotides were then annealed to single-stranded DNA template (2 μl single-stranded plasmid, 1 μl oligonucleotide, 1 μl 10×TM buffer, 6 μl water; heat at 94° C. for 2 minutes, 50° C. for 5 minutes, cool to room temperature). Double-stranded DNA was then prepared from the annealed oligonucleotide/template (add 2 μl 10×TM buffer, 2 μl 2.5 mM dNTPs, 1 μl 100 mM DTT, 1.5 μl 10 mM ATP, 4 μl water, 0.4μ; T7 DNA polymerase, 0.6 μl T4 DNA ligase; incubate at room temperature for two hours). E. coli (XL1 blue, Stratagene) was then transformed with the double-stranded DNA (1 μl double-stranded DNA, 10 μl 5×KCM, 40 μl water, 50 μl DMSO competent cells; incubate 20 minutes at 4° C., 10 minutes at room temperature), plated onto LB/agar containing 100 μg/ml ampicillin, and incubated at 37° C. overnight. Approximately four colonies from each plate were used to inoculate 5 ml 2YT containing 100 μg/ml ampicillin; these cultures were grown at 37° C. for 18-24 hours. Plasmids were then isolated from the cultures using Qiagen miniprep kit. These plasmids were sequenced to determine which IL1R2D-FB clones contained the desired mutation.

Sequencing of IL1R2D genes was accomplished as follows. The concentration of plasmid DNA was quantitated by absorbance at 280 nm. 800 ng of plasmid was mixed with sequencing reagents (8 μl DNA, 3 μl water, 1 μl sequencing primer, 8 μl sequencing mixture with Big Dye [Applied Biosystems]). The sequencing primers used were FB Forward and FB Reverse, shown below.

| | | |
|---|---|---|
| FB Forward | TATTCCGGATTATTCATACC | SEQ ID NO:44 |
| FB Reverse | CCTCTACAAATGTGGTATGGC | SEQ ID NO:45 |

The mixture was then run through a PCR cycle (96° C., 10 s; 50° C., 5 s; 60° C. 4 minutes; 25 cycles) and the DNA reaction products were precipitated (20 μl mixture, 80 μl 75% isopropanol; incubated 20 minutes at room temperature, pelleted at 14 K rpm for 20 minutes; wash with 250 μl 70% ethanol; heat 1 minute at 94° C.). The precipitated products were then suspended in Template Suppression Buffer (TSB, Applied Biosystems) and the sequence read and analyzed by an Applied Biosystems 310 capillary gel sequencer. In general, 3 out of 4 of the plasmids contained the desired mutation. A listing of the constructs and their mutant(s) is given below, although any cysteine mutants can be made in any of the given contexts.

| Construct | Mutant(s) |
|---|---|
| sIL1R2D-N83H-FB | E11C, I13C, V16C, Q108C, I110C, K112C, |
| | K114C, V117C, V124C, Y127C, E129C |
| sIL1R2D-N83H-FBHT | E11C, I13C, V16C, Q108C, I110C, K112C, |
| | Q113C, K114C, V117G, V124C, Y127C, |
| | E129C |
| sIL1R2D-N176H-FB | E11C |
| sIL1R2D-N176H-FBHT | E11C, V16C, V124C, E129C |
| sIL1R2D2M-FB | E11C, K12C, I13C, A107C, K112C, |
| | V124C, Y127. |

Mutagenic Oligonucleotides

| | | |
|---|---|---|
| E11C | TAAAATTATTTTACATTCACGTTCC | SEQ ID NO:46 |
| K12C | CACTAAAATTATACATTCTTCACGTTC | SEQ ID NO:47 |
| 113C | TGACACTAAAATACATTTTTCTTCACG | SEQ ID NO:48 |
| V16C | ATTTGCAGATGAACATAAAATTATTT | SEQ ID NO:49 |
| A107C | AAATATGGCTTGGCAATTATAACATAAG | SEQ ID NO:50 |
| Q108C | CTTAAATATGGCGCATGCATTATAACA | SEQ ID NO:51 |
| 1110C | GTTTCTGCTTAAAGCAGGCTTGTGCATT | SEQ ID NO:52 |
| K112C | GGGTAGTTTCTGACAAAATATGGC | SEQ ID NO:53 |
| Q113C | AACGGGTAGTTTACACTTAAATATGGC | SEQ ID NO:54 |
| K114C | CTGCAACGGGTAGGCACTGCTTAAATATG | SEQ ID NO:55 |
| V117C | CTCCGTCTCCTGCACAGGGTAGTTTCTG | SEQ ID NO:56 |
| V124C | CATATAAGGGCAACAAAGTCCTCC | SEQ ID NO:57 |
| Y127C | AAAAAACTCCATACAAGGGCACACAAG | SEQ ID NO:58 |
| E129C | TTTAAAAAAACACATATAAGGGCA | SEQ ID NO:59 |

Expression of IL-1 R1 Mutant Proteins

All IL1R-FB/FBHT plasmids were site-specifically transposed into the baculovirus shuttle vector (bacmid) by transforming the plasmids into DH10bac (Gibco/BRL) competent cells as follows: 1 μl DNA at 5 ng/μl, 10 μl 5×KCM [0.5 M KCl, 0.15 M CaCl₂, 0.25 M MgCl₂], 30 μl water was mixed with 50 μl PEG-DMSO competent cells, incubated at 4° C. for 20 minutes, 25° C. for 10 minutes, add 900 μl SOC and incubate at 37° C. with shaking for 4 hours, then plated onto LB/agar plates containing 50 μg/ml kanamycin, 7 μg/ml gentamycin, 10 μg/ml tetracycline, 100 μg/ml Bluo-gal, 10 μg/ml IPTG. After incubation at 37° C. for 24 hours, large white colonies were picked and grown in 3 ml 2YT media overnight. Cells were then isolated and double-stranded DNA was extracted from the cells as follows: pellet was resuspended in 250 μl of Solution 1 [15 mM Tris-HCl (pH 8.0), 10 mM EDTA, 100 μg/ml RNase A]. 250 μl of Solution 2 [0.2 N NaOH, 1% SDS] was added, mixed gently and incubated at room temperature for 5 minutes. 250 μl 3 M potassium acetate was added and mixed, and the tube placed on ice for 10 minutes. The mixture was centrifuged 10 minutes at 14,000×g and the supernatant transferred to a tube containing 0.8 ml isopropanol. The contents of the tube were mixed and placed on ice for 10 minutes; centrifuged 15 minutes at 14,000×g. The pellet was washed with 70% ethanol and air-dried and the DNA resuspended in 40 μl TE.

The bacmid DNA was used to transfect Sf9 cells. Sf9 cells were seeded at 9×10⁵ cells per 35 mm well in 2 ml of Sf-900 II SFM medium containing 0.5× concentration of antibiotic-antimycotic and allowed to attach at 27° C. for 1 hour. During this time, 5 μl of bacmid DNA was diluted into 100 μl of medium without antibiotics, 6 μl of CellFECTIN reagent was diluted into 100 μl of medium without antibiotics and then the 2 solutions were mixed gently and allowed to incubate for 30 minutes at room temperature. The cells were washed once with medium without antibiotics, the medium was aspirated and then 0.8 ml of medium was added to the lipid-DNA complex and overlaid onto the cells. The cells were incubated for 5 hours at 27° C., the transfection medium was removed and 2 ml of medium with antibiotics was added. The cells were incubated for 72 hours at 27° C. and the virus was harvested from the cell culture medium.

The virus was amplified by adding 0.5 ml of virus to a 50 ml culture of Sf9 cells at 2×10⁶ cells/ml and incubating at 27° C. for 72 hours. The virus was harvested from the cell culture medium and this stock was used to express the various IL1R constructs in High-Five cells. A 1 L culture of High-Five cells at 1×10⁶ cells/ml was infected with virus at an approximate MOI of 2 and incubated for 72 hours. Cells were pelleted by centrifugation and the supernatant was loaded onto an IL1R antagonist column at 1 ml/min, washed with PBS followed by a wash with Buffer A (0.2 M NaOAc pH 5.0, 0.2 M NaCl). The protein was eluted from the column by running a gradient from 0-100% of Buffer B (0.2 M NaOAc pH 2.5, 0.2 M NaCl) in 10 minutes followed by 15 minutes of 100% Buffer B at 1 mL/min collecting 2 mL fractions in tubes containing 300 μl of unbuffered Tris. The appropriate fractions were pooled, concentrated and dialyzed against 5 L of 50 mM Tris pH 8.0, 100 mM NaCl at 4° C. and filtered through a 0.2 μm filter.

### Example 3

Protocol for Determining Statistical Significance of Differences Between Hits and a Library

Statistical Analysis of Chemical Substructures in Tether Hit Compounds

The following analysis was performed to determine whether features found in selected tethers differ from the representation of those features in the overall screening library in a statistically significant fashion.

Significance

Given a starting population in which fraction X members have a given property and fraction (1-X) do not, how likely is it that a sample of size N with Y members possessing that same property could be selected purely at random?

The answer can be found by discrete integration of the binomial distribution for a sample of size N:

B(N,Y,X)=(N!/(Y!(N−Y)!))*(X\hat{ }Y)*((1-X)A(N−Y))

For large N, the normal distribution can be used to approximate the binomial result.

The significance value (p) is determined by summing up the combined probability of all results with a higher likelihood of occurrence than the result, which actually occurred. If Y<(N/2), then

p=1.0−[Sum B(N,i,X) for i over the interval Y+1 to (N−Y)−1].

If Y>(N/2) then

p=1.0−[Sum B(N,i,X) for i over the interval (N−Y)+1 to Y−1].

The objective is to rule out from consideration the vast majority all possible random events. If p≦0.01 (−log p≧2), then there is only a 1% chance that the result could have occurred randomly. If p≦0.0001 (−log p≧4). Then there is only a 0.01% chance that the result could have been random.

Enrichment Ratios

Once a given significant threshold has been applied to filter out non-significant results, the enrichment ratio is computed by dividing the fraction of sample members (Y/N) possessing a given property by the fraction present in the overall population (X). The depletion ratio is simply the inverse.

enrichment ratio=(YIN)/X

depletion ratio=X/(Y/N)

### Example 4

Characterization of Interleukin-1 Receptor Type 1 Molecular Interaction Site

Binding of the IL-1 receptor (accession number SWS P14778) to IL-1alpha or IL-1beta is another important mediator of immune and inflammatory responses. This interaction is controlled by at least three mechanisms. Firstly, the protein IL-R2 binds to IL-1alpha and IL-1beta but does not signal. Secondly, proteolytically processed IL-1R1 and IL-1R2 are soluble and bind to IL-1 in circulation. Finally there exists a natural IL-1R antagonist, IL-1Ra, that functions by binding IL-1R1 and thereby blocking IL-1R1 binding of IL-1alpha and IL-1beta. Inhibition of these interactions with an orally available small molecule would be desirable in treatment of diseases such as rheumatoid arthritis, autoimmune disorders, and ischemia. Two structures of IL-1 R1 have been solved [with a antagonist peptide, IGOY, Vigers, G. P. A., et al., J. Biol. Chem. 275:36927-36933 (2000); with receptor antagonist, 1IRA, Schreuder, H., et al., Nature 386: 194-200 (1997)].

From the two crystal structures, it appears that there are two binding pockets of interest, one is fixed between the two structures, and the other is adaptable. The fixed region comprises a shallow glutamine-binding pocket and a tyrosine surface. Each of the known ligands for IL-1R1 makes contacts with this portion of the protein surface, and the contacts are essential for specificity of the respective binding interactions. The shallow pocket appears to be preorganized around a beta strand. The adaptable region comprises the 110's loop and a hydrophobic pocket.

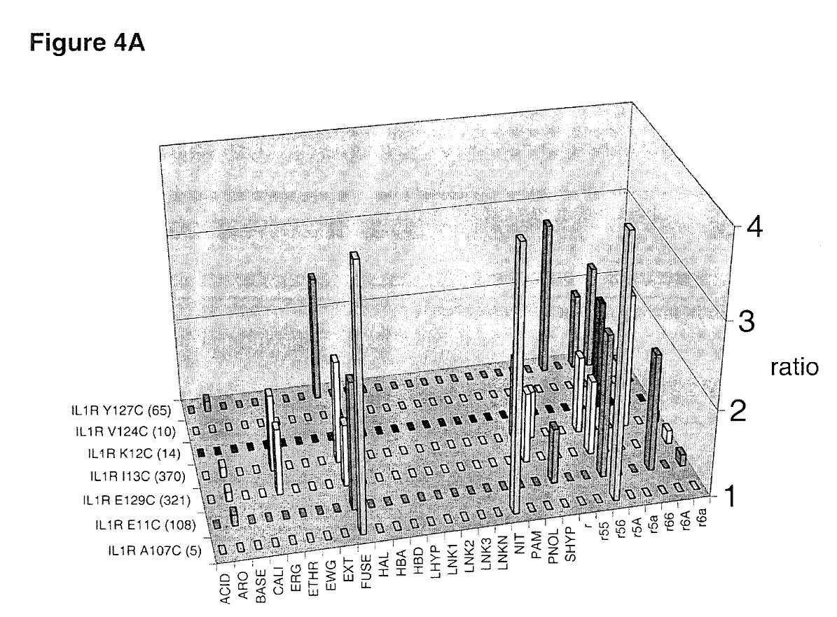

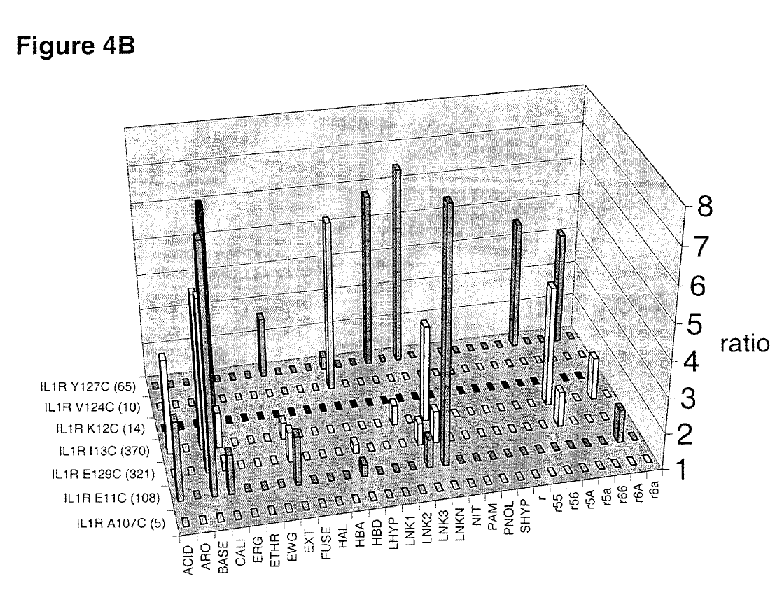

Several cysteine mutants of IL-1R1 expressed from the sIL1R2D-N83H-FB construct (see Example 2) were tested in order to probe the aforementioned binding regions, including E11C, K12, I13C, A107C, V124C, Y127C, and E129C. In this analysis, detectable formation of a given protein-compound conjugate determined whether the corresponding compound was scored as a hit. The A107, V124C, and Y127C IL-1R1 mutants did not show an appreciable number of hits, whereas the remaining residues did show an appreciable number of hits. The hits of each of the mutants were categorized according to whether they possessed a certain chemical feature, and significant differences between the prevalence of the chemical feature among hits as compared with the prevalence among the total library were calculated. Significant enrichments and significant depletions are shown in FIGS. 4A and 4B, respectively. No significant enrichment of primary amines is observed for IL-1R1, as was the case for many mutants of HIV Integrase (vide supra). On the other hand, modest significant enrichments for 66 fused ring systems were observed for the E11C, I13C, and Y127C mutants (FIG. 4A), and were not observed for any of the HIV integrase mutants examined. Thus the molecular interaction sites on these polypeptides demonstrate target-specific differences.

Subtle differences are observed for patterns of enrichment even among residues at the same site on the same target otherwise showing similar characteristics. For example, significant enrichment of fused ring systems (FUSE), and particularly of 56 fused ring systems, is observed for the E11C, I13C, Y127C and E129C mutants (FIG. 4A). However, unlike the other three mutants of this group, E129C does not demonstrate any significant enrichment for 66 fused ring systems. The difference is also reflected in the fact that E129C shows a somewhat lower ratio for enrichment of fused ring systems. These types of differences between mutants can be understood because the residues have different positions within a site of interest; therefore a ligand candidate tethered to the different residues will not be necessarily accessing the site of interest in an identical manner.

Significant depletion of basic groups with a high ratio is observed for the IL-1R1 E11C, I13C, and E129; the depletion ratios are 8, 5, and 6, respectively (FIG. 4B). Additionally, significant depletion of two rings linked by more than three atoms is observed at E11C, I13C, Y127C and E129C.

As mentioned above, the I13C and E129C residues show enrichments of 56 fused ring systems. These same residues also show a depletion of 5-membered aliphatic rings (FIG. 4B). Taken together, these results suggest that the binding site comprising these two residues is characterized by a preference for 56 fused ring systems comprising a 5-membered ring that is at least partially unsaturated.

In order to obtain statistical information, it is preferable to obtain information from a sizeable number of hits. Thus, as mentioned above, any ligand conjugate producing detectable formation of the protein-compound conjugate is counted as a hit. Techniques such as mass spectrometry may detect as little as 1% conjugation. In another variation of the method, a specific fraction of polypeptide forming the reversible covalent bond, e.g., 30% or higher is counted as a hit, whereas any compounds conjugating less than 30% are not counted as a hit. In general, if a protein is capable of reacting to the extent of 50% or greater with the ligand candidate, it is considered to be a strong hit. If the extent of reaction lies in the 20-50 percent range it is considered a moderate hit. If the extent of reaction is less than 20%, it is considered a weak hit. In one embodiment of the methods described herein, a specific fraction of protein forming the reversible covalent bond may be used as a threshold value. In another embodiment of the methods herein, statistical significance of enrichments and depletions are calculated using only the moderate to strong hits.

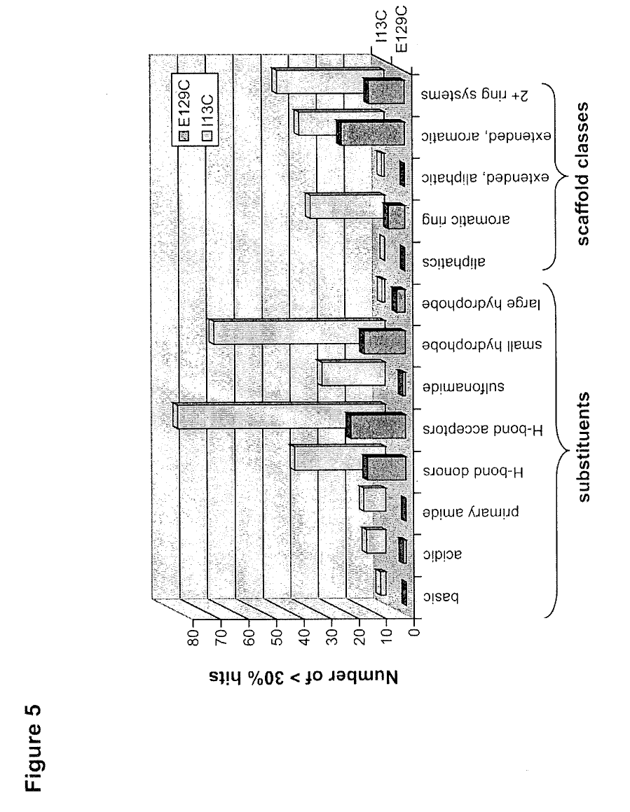

FIG. 5 shows an analysis of the number of moderate to strong hits (<30% fraction conjugated) obtained for the IL-1R1 mutants I13C and E129C. Elements categorized in FIG. 5 under scaffolds classes correspond to presence of either a molecular framework or a substructure, depending upon the specific compound. There are some similar themes observed as compared with the statistical enrichment and depletion patterns shown in FIG. 4. For example, in FIG. 5, it can be observed that there are a large number (˜60) of compounds with small hydrophobic groups that form a conjugate with the I13C mutant, whereas far fewer (˜10) form a conjugate with the E129C mutant. Likewise, in FIG. 4A, it may be observed that E129C shows a significant enrichment for small hydrophobic groups, whereas the E129C mutant does not show enrichment for small hydrophobic groups. Similar consistencies may be observed for depletions. For example, FIG. 5 shows no compounds with basic groups conjugating to E129C and very few (<5) conjugating to I13C. Likewise, in FIG. 4B, both I13C and E129C show significant depletions with high ratios of ligand candidates comprising basic groups. However, although numerous compounds with hydrogen bond acceptors conjugate to I13C, for example, no statistical enrichments of hydrogen bond acceptors is found in FIG. 4A for I13C, or any other residue of IL-1R1. Therefore, the information obtained from these two types of analyses is complementary, with one examining a large number of relatively weaker hits, and the other using a smaller number of relatively stronger hits.

The entire disclosure of all documents cited throughout this application are incorporated herein by reference.

What is claimed is:

1. A method for characterizing a molecular interaction site on a protein, the method comprising:
a) contacting a polypeptide comprising a first chemically reactive group at a site of interest with a library of ligand candidates wherein each ligand candidate comprises a second chemically reactive group that is capable of forming a reversible covalent bond with the first chemically reactive group;
b) forming a reversible covalent bond between the first chemically reactive group and the second chemically reactive group of a plurality of the ligand candidates;
c) detecting the ligand candidates that formed the reversible covalent bond;
d) identifying a physicochemical property of at least one of the ligand candidates that formed the reversible covalent bond;
e) determining the number of ligand candidates that formed the reversible covalent bond having the physicochemical property; and
f) determining the number of ligand candidates in the library having the physicochemical property.

2. The method of claim 1 further comprising:
comparing the prevalence of ligand candidates with the physicochemical property that form the reversible covalent bond with the prevalence of ligand candidates with the physicochemical property in the library and measuring the statistical significance of the comparison.

3. The method of claim 2 further comprising:
characterizing the molecular interaction site by a significant enrichment of the prevalence of the physicochemical property among ligand candidates that formed the reversible covalent bond.

4. The method of claim 2 further comprising:
characterizing the molecular interaction site by a significant depletion of the prevalence of the physicochemical property among ligand candidates that formed the reversible covalent bond.

5. The method of claim 1 wherein the protein is individually contacted with members of the library of ligand candidates.

6. The method of claim 1 wherein the site of interest is an active site, an allosteric site, a small molecule binding site, or a protein-protein binding site.

7. A method for characterizing a molecular interaction site on a protein, the method comprising:
a) contacting a polypeptide comprising a first chemically reactive group at a site of interest with a library of ligand candidates wherein each ligand candidate comprises a second chemically reactive group that is capable of forming a reversible covalent bond with the first chemically reactive group;
b) forming a reversible covalent bond between the first chemically reactive group and the second chemically reactive group of a plurality of the ligand candidates;
c) detecting the ligand candidates that formed the reversible covalent bond;
d) identifying a physicochemical property of at least one of the ligand candidates that formed the reversible covalent bond;
e) calculating the statistical significance of the incidence of ligand candidates having the physicochemical property in the plurality; and
f) repeating each of step a) through step e) for at least one positional variant of the polypeptide, the positional variant comprising an identical first chemically reactive group at a different sequence location.

8. The method of claim 7 wherein at least the formation of the reversible covalent bond is performed in the presence of a reducing agent.

9. The method of claim 1 wherein the physicochemical property of the ligand candidate is a molecular property selected from the group consisting of molecular weight, molar refractivity, LogP, polar surface area, total number of rotatable bonds, total number of aromatic atoms, total number of hydrogen bond donors and total number of hydrogen bond acceptors.

10. The method of claim 1 wherein the physicochemical property of the ligand candidate is the presence of a chemical feature selected from the group consisting of a molecular shape, an atomic skeleton and a peripheral functionality.

11. The method of claim 10 wherein the molecular shape is selected from the group consisting of:

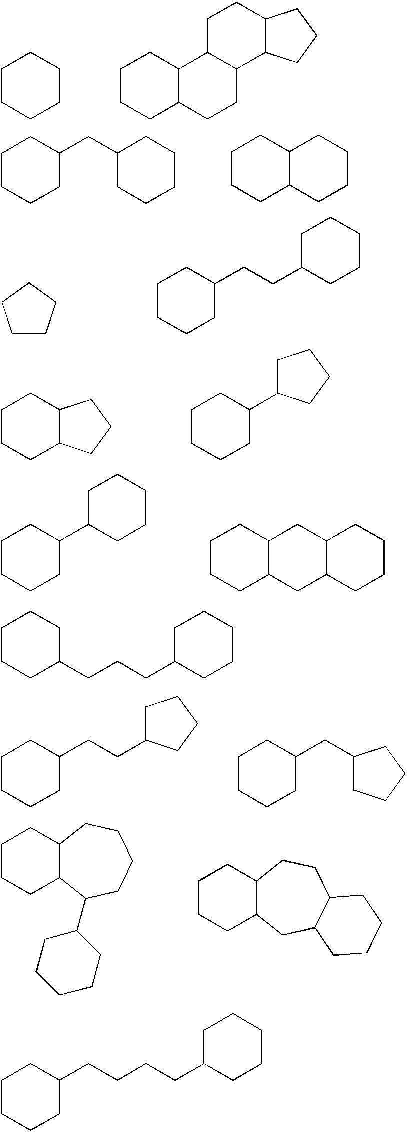

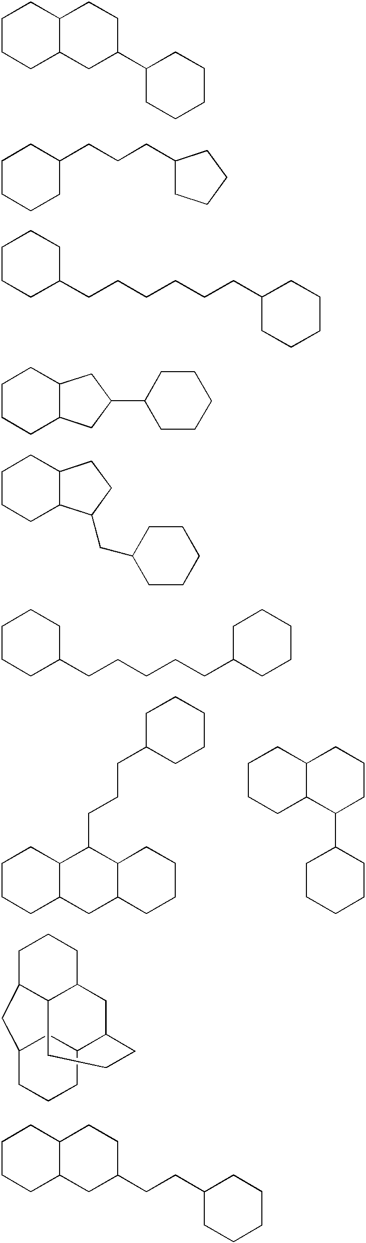

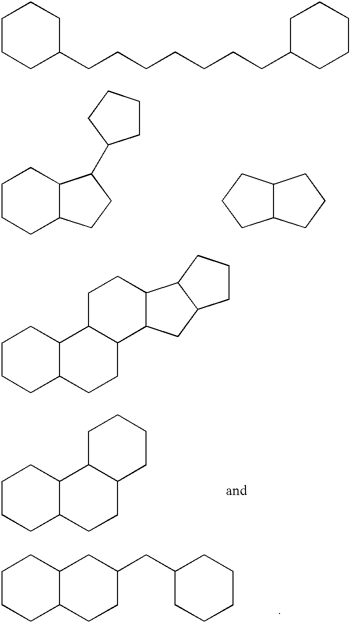

12. The method of claim 10 wherein the atomic skeleton is selected from the group consisting of:

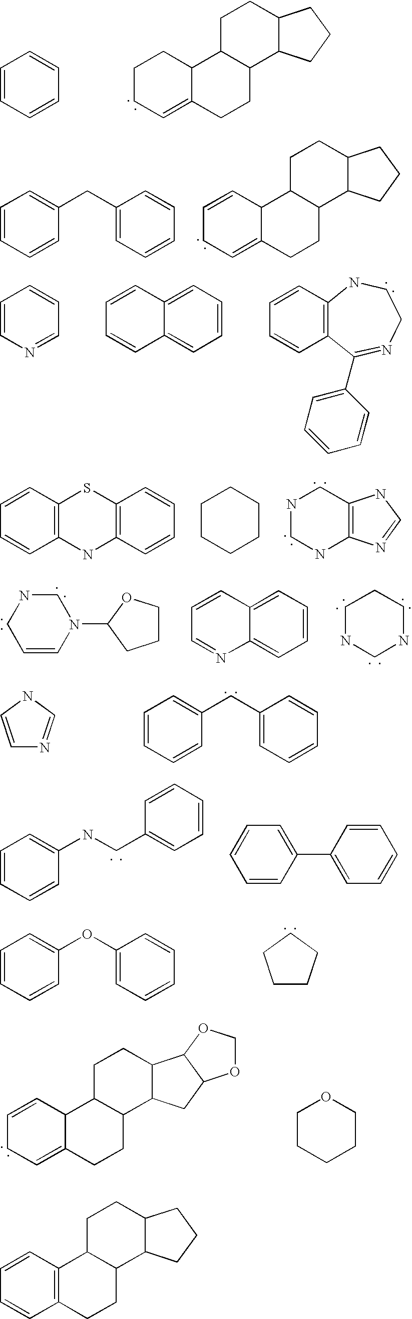

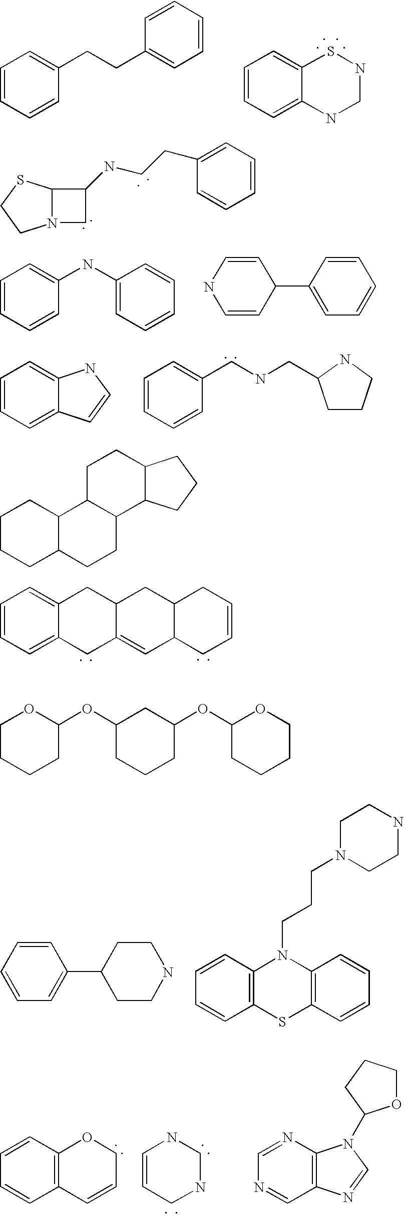

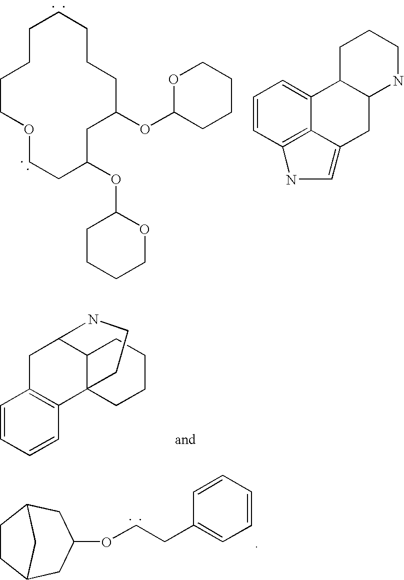

13. The method of claim 10 wherein the peripheral functionality is selected from the group consisting of:

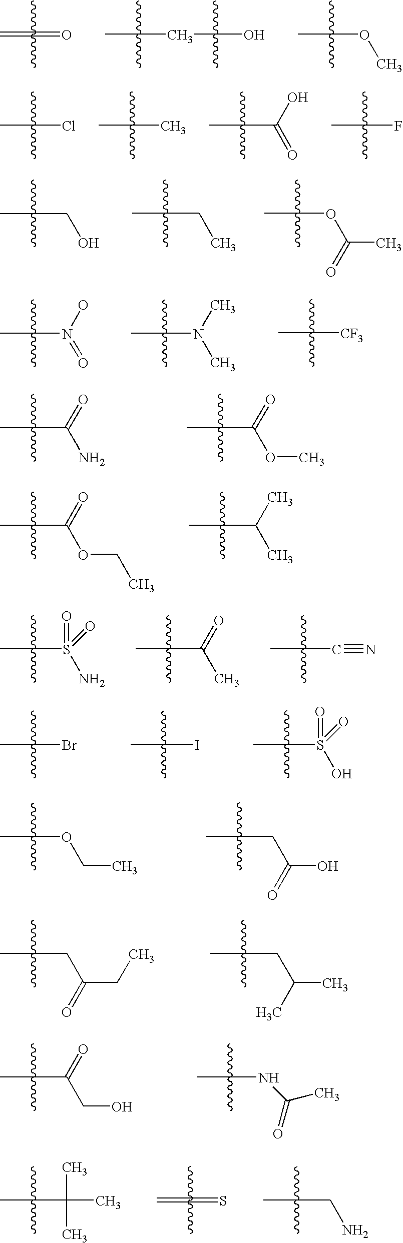

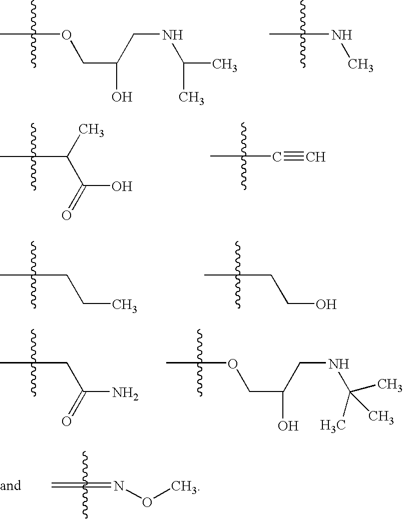

14. The method of claim 10 wherein the peripheral functionality is selected from the group consisting of:

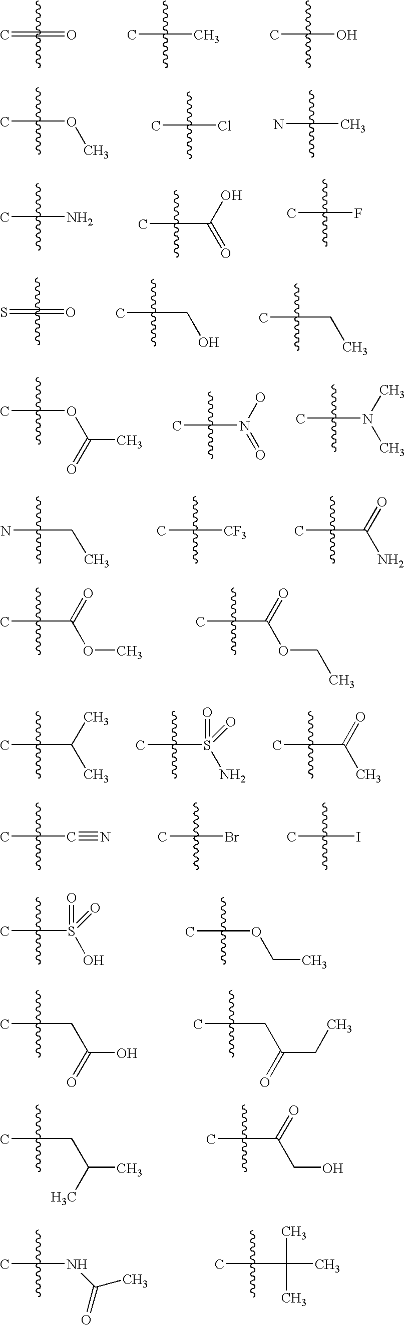

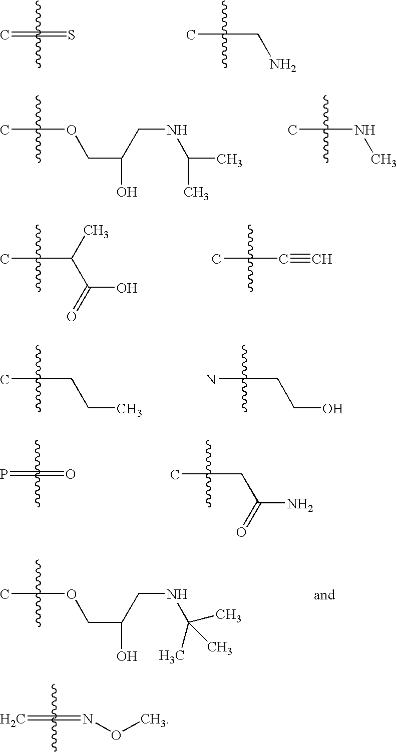

15. The method of claim 10 wherein the physicochemical property is the presence of a chemical feature selected from the group consisting of a nitro, an amine, an amide, a sulfonamide, a carboxylic acid, an alcohol, a phenol, a halogen, an aryl halide and an ether.

16. A method for characterizing a molecular interaction site on a protein, the method comprising:
a) contacting a polypeptide comprising a thiol or a masked thiol at a site of interest with a library of ligand candidates wherein each ligand candidate comprises a disulfide group that is capable of engaging in disulfide exchange to form a disulfide linkage between the polypeptide and the ligand candidate;
b) forming the disulfide linkage between the polypeptide and a plurality of the ligand candidates;
c) detecting the ligand candidates that formed the disulfide linkage;
d) identifying a physicochemical property of at least one of the ligand candidates that formed the disulfide linkage; and
e) calculating the statistical significance of the incidence of ligand candidates having the physicochemical property in the plurality;
wherein the physicochemical property is selected from the group consisting of molecular weight, LogP, number of hydrogen bond donors, and number of hydrogen bond acceptors.
